(19) United States
(12) Patent Application Publication
(10) Pub. No.: US 2006/0111350 A1
(43) Pub. Date: May 25, 2006

(54) Solid forms of linezolid and processes for preparation thereof

(76) Inventors: Judith Aronhime, Rehovot (IL); Tamas Koltai, Natania (IL); Viviana Braude, Kadima (IL); Serguei Fine, Qiriat-Arbaa (IL); Tamar Niddam, Yehud (IL)

Correspondence Address: KENYON & KENYON LLP, ONE BROADWAY, NEW YORK, NY, 10004, US

(21) Appl. No.: 11/171,579

(22) Filed: Jun. 29, 2005

Related U.S. Application Data
(60) Provisional application No. 60/584,371, filed on Jun. 29, 2004.
(60) Provisional application No. 60/584,283, filed on Jun. 30, 2004.
(60) Provisional application No. 60/601,086, filed on Aug. 12, 2004.
(60) Provisional application No. 60/602,227, filed on Aug. 17, 2004.
(60) Provisional application No. 60/633,887, filed on Dec. 7, 2004.
(60) Provisional application No. 60/678,440, filed on May 5, 2005.
(60) Provisional application No. 60/684,410, filed on May 24, 2005.

(51) Int. Cl. A61K 31/5377; C07D 413/02

(52) U.S. Cl. 514/235.2; 544/137

(57) ABSTRACT

Novel crystalline forms of Linezolid, designated as Form TIII, Form V, Form VI, Form IX, Form X, Form XII, Form XIV, Form XVII, and Form XVIII, are disclosed. The novel crystalline forms are characterized by powder X-ray diffraction, FTIR and FTRaman spectroscopy, and differential scanning calorimetry. Methods of preparing the novel crystalline forms, pharmaceutical compositions comprising the novel crystalline forms, and methods of using the novel crystalline forms to treat gram positive bacterial infections are also described. Amorphous Linezolid is also disclosed.

### CROSS-REFERENCE TO RELATED APPLICATIONS

This application claims the benefit of provisional applications Ser. No. 60/584,371, filed Jun. 29, 2004; 60/584,283, filed Jun. 30, 2004; 60/601,086, filed Aug. 12, 2004; 60/602,227, filed Aug. 17, 2004; 60/633,887, filed Dec. 7, 2004; 60/678,440, filed May 5, 2005; and 60/684,410, filed May 24, 2005; which are incorporated herein by reference.

### FIELD OF THE INVENTION

The present invention relates to the solid state chemistry of Linezolid and provides novel crystalline and amorphous forms of Linezolid.

### BACKGROUND OF THE INVENTION

Linezolid [(S)—N-[[3-(3-Fluoro-4-morpholinyl)phenyl]-2-oxo-5-oxazolidinyl]methyl]acetamide] is an antimicrobial agent. Linezolid is an oxazolidinone, having the following structure:

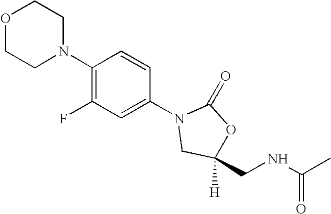

Linezolid is described in the Merck index (13th edition, Monograph number: 05526, CAS Registry Number: 165800-03-3) as white crystals, mp 181.5-182.5°. Linezolid, as well as a process for its preparation, is disclosed in U.S. Pat. No. 5,688,792 (example 5). Linezolid produced had a m.p. of 181.5-182.5°.

U.S. Pat. No. 6,559,305 and U.S. Pat. No. 6,444,813 disclose a new crystal form (Form II) of Linezolid. According to U.S. Pat. No. 6,559,305, Form II differs from Form I in its IR spectrum, X-ray powder diffraction spectrum and melting point. According to U.S. Pat. No. 6,559,305, at column 3, line 37: “Crystal Form II is the most stable form below about 85°.”

The present invention relates to the solid state physical properties of Linezolid. These properties can be influenced by controlling the conditions under which Linezolid is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. These conformational and orientation factors in turn result in particular intramolecular interactions such that different polymorphic forms may give rise to distinct spectroscopic properties that may be detectable by powder X-ray diffraction, solid state ¹³C NMR spectrometry and infrared spectrometry. A particular polymorphic form may also give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional polymorphic forms of Linezolid.

### SUMMARY OF THE INVENTION

The present invention discloses solid crystalline and amorphous forms of (S)—N-[[3-[3-Fluoro-4-(4-morpholinyl)phenyl]-2-oxo-5-oxazo-lidinyl]methyl]-acetamide and a racemic mixture of solid crystal form (S) and (R)—N-[[3-[3-Fluoro-4-(4-morpholinyl)phenyl]-2-oxo-5-oxazo-lidinyl]methyl]-acetamide.

One embodiment of the present invention is crystalline Linezolid racemate.

Another embodiment of the present invention is Linezolid hydrate.

The present invention relates to novel solid crystal forms of (S)—N-[[3-[3-Fluoro-4-(4-morpholinyl)phenyl]-2-oxo-5-oxazo-lidinyl]methyl]-acetamide (Linezolid), referred to herein as Form TIII, Form V, Form VI, Form IX, Form X, Form XII, Form XIV, Form XVII, and Form XVIII. The crystalline forms of Linezolid described herein have the powder X-ray diffraction (PXRD), Fourier transform infrared spectroscopy (FTIR), FTRaman, or differential scanning calorimetry (DSC) characteristics described herein.

A particular embodiment of the present invention is crystalline Linezolid Form TIII, characterized by a PXRD pattern with peaks at about 13.5, 16.8, 21.1, 21.7, and 22.2±0.2 degrees 2 theta.

Another embodiment of the present invention is crystalline Linezolid Form V, characterized by a PXRD pattern with peaks at about 12.3, 17.6, 22.2, 24.6, and 31.8±0.2 degrees 2 theta.

Another embodiment of the present invention is crystalline Linezolid Form VI, characterized a PXRD pattern with peaks at about 12.3, 21.3, 24.7, 25.2, and 27.7±0.2 degrees 2 theta.

Another embodiment of the present invention is crystalline Linezolid Form IX, characterized by a PXRD pattern with peaks at about 13.4, 17.9, 21.4, 22.3, and 25.6±0.2 degrees 2 theta. This form is a racemate.

Another embodiment of the present invention is crystalline Linezolid Form X, characterized by a PXRD pattern with peaks at about 4.7, 15.7, and 21.7±0.2 degrees 2 theta.

Another embodiment of the present invention is crystalline Linezolid Form XII, characterized by a PXRD pattern with peaks at about 10.4, 10.7, 17.1, and 22.7±0.2 degrees 2 theta.

Another embodiment of the present invention is crystalline Linezolid Form XIV, characterized by a PXRD pattern with peaks at about 3.7, 5.0, 15.8, and 16.7±0.2 degrees 2 theta.

Another embodiment of the present invention is crystalline Linezolid Form XVII, characterized by a PXRD pattern with peaks at about 6.1, 12.3, 18.4, and 21.2±0.2 degrees 2 theta.

Another embodiment of the present invention is crystalline Linezolid Form XVIII, characterized by a PXRD pattern with peaks at about 6.0, 11.8, 17.2, 18.2, and 24.9±0.2 degrees 2 theta.

The PXRD peaks in degrees 2 theta of the novel crystalline forms are shown in Table 1, with the most characteristic peaks indicated in bold.

TABLE 1

| Form | Form | Form | Form | Form | Form | Form | Form | Form |
|---|---|---|---|---|---|---|---|---|
| TIII | V | VI | IX | X | XII | XIV | XVII | XVIII |
| 7.4 | 7.5 | 12.3 | 7.3 | 3.5 | 10.4 | 3.7 | 6.1 | 6.0 |
| 13.5 | 12.3 | 17.5 | 9.3 | 4.7 | 10.7 | 5.0 | 11.0 | 11.3 |
| 14.3 | 13.5 | 21.3 | 13.4 | 10.3 | 14.5 | 15.8 | 11.7 | 11.8 |
| 16.8 | 17.6 | 22.2 | 17.9 | 15.7 | 17.1 | 16.7 | 12.3 | 13.0 |
| 18.1 | 21.1 | 24.7 | 18.6 | 20.2 | 21.9 | | 13.0 | 17.2 |
| 21.1 | 22.2 | 25.2 | 21.4 | 21.7 | 22.7 | | 14.4 | 18.2 |
| 21.7 | 24.6 | 27.7 | 22.3 | | 23.8 | | 18.4 | 19.0 |
| 22.2 | 25.5 | 28.1 | 25.6 | | | | 21.2 | 23.1 |
| 23.6 | 27.8 | 33.2 | 29.3 | | | | 22.2 | 24.9 |
| 25.5 | 31.8 | 35.2 | | | | | | 25.5 |

The characteristic FTIR peaks of Form II (listed in U.S. Pat. No. 6,444,813), Form TIII, Form V, Form VI, Form IX, Form X and Form XII in cm⁻¹ are shown in Table 2a. This data was obtained using a Perkin Elmer SPECTRUM ONE FT-IR spectrometer in DRIFT mode. The samples in the 4000-400 cm⁻¹ interval were scanned 16 times with 4.0 cm^{31 1} resolution.

TABLE 2a

| Form | Form | Form | Form | Form | Form | Form |
|---|---|---|---|---|---|---|
| II | TIII | V | VI | IX | X | XII |
| 3364 | 1535 | 3336 | 3334 | 3451 | 3090 | 3212 |
| 1748 | 1254 | 2772 | 2770 | 3344 | 3290 | 3364 |
| 1675 | | 2497 | 2496 | 1735 | 2955 | 2853 |
| 1537 | | 1742 | 1741 | 1427 | 2858 | 1744 |
| 1517 | | 1662 | 1662 | 1082 | 1524 | 1675 |
| 1445 | | 1542 | 1545 | 687 | 1335 | 678 |
| 1410 | | 1516 | 1541 | 1515 | 1195 | 663 |
| 1401 | | 1425 | 1516 | 599 | 1115 | |
| 1358 | | 1335 | 1333 | 467 | 1081 | |
| 1329 | | 1229 | 1228 | | 940 | |
| 1287 | | 1176 | 1174 | | 927 | |
| 1274 | | 1038 | 1036 | | 802 | |
| 1253 | | | | | 752 | |
| 1237 | | | | | | |
| 1221 | | | | | | |
| 1145 | | | | | | |
| 1130 | | | | | | |
| 1123 | | | | | | |
| 1116 | | | | | | |
| 1078 | | | | | | |
| 1066 | | | | | | |
| 1049 | | | | | | |
| 907 | | | | | | |
| 852 | | | | | | |
| 758 | | | | | | |

The characteristic FTIR peaks of Form II (listed in U.S. Pat. No. 6,444,813), Form TIII, Form V, Form VI, Form IX, Form X and Form XII in cm⁻¹ are shown in Table 2b. This data was obtained using a Perkin Elmer SPECTRUM ONE FT-IR spectrometer using mineral oil mull technique. The samples in the 4000-400 cm⁻¹ interval were scanned 16 times with 4.0 cm⁻¹ resolution.

TABLE 2b

| Form | Form | Form | Form | Form | Form | Form |
|---|---|---|---|---|---|---|
| II | TIII | V | VI | IX | X | XII |
| 3364 | 1535 | 3334 | 3334 | 3451 | 3090 | 3212 |
| 1748 | 1254 | 2496 | 2496 | 3344 | 1524 | 663 |
| 1675 | | 1741 | 1741 | 1735 | 1335 | |
| 1537 | | 1662 | 1662 | 1427 | 1195 | |
| 1517 | | 1545 | 1545 | 1082 | 1115 | |
| 1445 | | 1516 | 1516 | 687 | 1081 | |
| 1410 | | 1228 | 1228 | | 940 | |
| 1401 | | 1036 | 1036 | | 927 | |
| 1358 | | | | | 802 | |
| 1329 | | | | | 752 | |
| 1287 | | | | | | |
| 1274 | | | | | | |
| 1253 | | | | | | |
| 1237 | | | | | | |
| 1221 | | | | | | |
| 1145 | | | | | | |
| 1130 | | | | | | |
| 1123 | | | | | | |
| 1116 | | | | | | |
| 1078 | | | | | | |
| 1066 | | | | | | |
| 1049 | | | | | | |
| 907 | | | | | | |
| 852 | | | | | | |
| 758 | | | | | | |

The characteristic FTRaman peaks of Form II, Form TIII, Form V, Form VI, and Form X in cm⁻¹ are shown in Table 3.

TABLE 3

| Form II | Form TIII | Form V | Form VI | Form X |
|---|---|---|---|---|
| 2853 | 724 | 2933 | 2996 | 2957 |
| 1673 | | 2978 | 2942 | 2859 |
| 1440 | | 1660 | 2498 | 975 |
| 1409 | | 1428 | 1037 | 880 |
| 1207 | | 1465 | 463 | 752 |
| 651 | | 1082 | | 715 |
| 432 | | 1036 | | |
| 142 | | 904 | | |
| | | 661 | | |
| | | 462 | | |
| | | 424 | | |
| | | 339 | | |

The present invention provides methods of preparing the novel crystalline Form TIII, Form V, Form VI, Form IX, Form X, Form XII, Form XIV, Form XVII, and Form XVIII of Linezolid.

One embodiment of the present invention is crystalline Linezolid racemate. Crystalline Linezolid racemate is a mixture of the (S) and (R) enantiomers of N-[[3-(3-Fluoro-4-morpholinyl)phenyl]-2-oxo-5-oxazolidinyl]methyl]acetamide.

Yet another embodiment of the present invention is an amorphous form of Linezolid and a method for preparing thereof.

A further embodiment of the present invention is pharmaceutical formulations comprising any one of the novel crystalline Form TIII, Form V, Form VI, Form IX, Form X, Form XII, Form XIV, Form XVII, and Form XVIII, as well as the amorphous form of Linezolid, and a pharmaceutically acceptable excipient.

Also provided is the method of treating gram positive bacterial infections using said pharmaceutical formulations.

The crystalline forms of the present invention may exist in anhydrous forms as well as in hydrated and solvated forms. The present invention encompasses solvates, particularly hydrates, of the novel crystalline forms of Linezolid described herein where those solvates or hydrates have the powder X-ray diffraction (PXRD), Fourier transform infrared spectroscopy (FTIR), FTRaman, or digital scanning calorimetry (DSC) characteristics described herein.

### FIGURES

FIG. 1 is a powder X-Ray diffractogram of crystalline Linezolid Form TIII.

FIG. 2a-2c is an FTIR spectrum of crystalline Linezolid Form TIII, obtained using mineral oil mull technique.

FIG. 2d-2f is an FTIR spectrum of crystalline Linezolid Form TIII, obtained using DRIFT technique.

FIG. 3 is a DSC thermogram of crystalline Linezolid Form TIII.

FIG. 4a-4d is an FTRaman spectrum of crystalline Linezolid Form TIII.

FIG. 5 is a powder X-Ray diffractogram of crystalline Linezolid Form V.

FIG. 6a-6c is an FTIR spectrum of crystalline Linezolid Form V, obtained using mineral oil mull technique.

FIG. 6d-6f is an FTIR spectrum of crystalline Linezolid Form V, obtained using DRIFT technique.

FIG. 7a-7d is an FTRaman spectrum of crystalline Linezolid Form V.

FIG. 8 is a powder X-Ray diffractogram of crystalline Linezolid Form VI.

FIG. 9a-9c is an FTIR spectrum of crystalline Linezolid Form VI, obtained using mineral oil mull technique.

FIG. 9d-9f is an FTIR spectrum of crystalline Linezolid Form VI, obtained using DRIFT technique.

FIG. 10a-10d is an FTRaman spectrum of crystalline Linezolid Form VI.

FIG. 11 is a powder X-Ray diffractogram of crystalline Linezolid Form IX.

FIG. 12a-12d is an FTIR spectrum of crystalline Linezolid Form IX, using DRIFT technique.

FIG. 13 is a powder X-Ray diffractogram of crystalline Linezolid Form X.

FIG. 14a-14c is an FTIR spectrum of crystalline Linezolid Form X, obtained using mineral oil mull technique.

FIG. 14d-14f is an FTIR spectrum of crystalline Linezolid Form X, obtained using DRIFT technique.

FIG. 15 is a DSC thermogram of crystalline Linezolid Form X.

FIG. 16a-16d is an FTRaman spectrum of crystalline Linezolid Form X.

FIG. 17 is a powder X-Ray diffractogram of crystalline Linezolid Form XII.

FIG. 18a-18d is an FTIR spectrum of crystalline Linezolid Form XII, obtained using DRIFT technique.

FIG. 19 is a powder X-Ray diffractogram of crystalline Linezolid Form XIV.

FIG. 20 is a powder X-Ray diffractogram of crystalline Linezolid Form XVII.

FIG. 21 is a powder X-Ray diffractogram of crystalline Linezolid Form XVIII.

FIG. 22 is a powder X-Ray diffractogram of amorphous Linezolid.

FIG. 23a-23c is an FTIR spectrum of amorphous Linezolid, obtained using mineral oil mull technique.

FIG. 24 is a DSC thermogram of amorphous Linezolid.

FIG. 25 is a DSC thermogram of crystalline Linezolid Form II.

FIG. 26 is a DSC thermogram of crystalline Linezolid Form V.

FIG. 27 is a DSC thermogram of crystalline Linezolid Form VI.

FIG. 28 is a DSC thermogram of crystalline Linezolid Form IX.

FIG. 29 shows the needle shape of crystals of crystalline linezolid Form II as seen through a microscope.

FIG. 30 shows the plate shape of crystals of crystalline linezolid Form TIII as seen through a microscope.

FIG. 31 shows the plate shape of crystals of crystalline linezolid Form V as seen through a microscope.

FIG. 32 shows the plate shape of crystals of crystalline linezolid Form VI as seen through a microscope.

FIG. 33 shows the plate shape of crystals of crystalline linezolid Form IX as seen through a microscope.

FIG. 34 shows the plate shape of crystals of crystalline linezolid Form X as seen through a microscope.

FIG. 35 shows the shape of crystals of crystalline linezolid Form XII as seen through a microscope.

### DETAILED DESCRIPTION OF THE INVENTION

As used herein, “room temperature” or “RT” is meant to indicate a temperature of about 18-25° C., preferably about 20-22° C.

“Therapeutically effective amount” means the amount of a crystalline form that, when administered to a patient for treating a disease or other undesirable medical condition, is sufficient to have a beneficial effect with respect to that disease or condition. The “therapeutically effective amount” will vary depending on the crystalline form, the disease or condition and its severity, and the age, weight, etc., of the patient to be treated. Determining the therapeutically effective amount of a given crystalline form is within the ordinary skill of the art and requires no more than routine experimentation.

The present invention provides linezolid hydrate. Also provided are crystalline forms of linezolid.

The previously known Linezolid Form II forms needle shaped crystals. The flowability of crystals with an irregular plate shape of the present invention is much better than the flowability of crystals with needle shape. Another advantage of irregular plate crystals is that they are easier to work with than needle shaped Linezolid of the prior art. The bulk properties of the irregular plate shape crystals are also advantageous compared to those of the prior art Linezolid.

Preferably, the crystalline forms of the present invnetion are in the irregular shape of plate crystals.

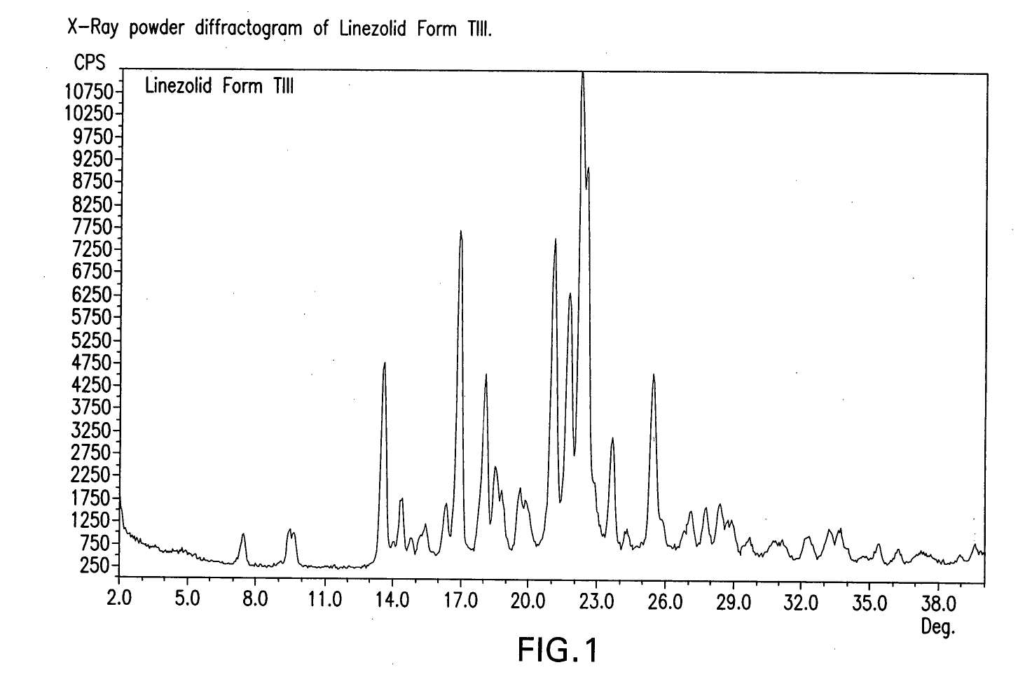

A particular embodiment of the present invention is a crystalline Linezolid (denominated Form TIII), characterized by a powder X-ray diffraction (PXRD) pattern with peaks at about 13.5, 16.8, 21.1, 21.7, and 22.2±0.2 degrees 2 theta. Crystalline Linezolid Form TIII may be further characterized by a PXRD pattern with peaks at about 7.4, 14.3, 18.1, 23.6, and 25.5±0.2 degrees 2 theta. Crystalline Linezolid Form TIII may be further characterized by a PXRD pattern substantially as depicted in FIG. 1.

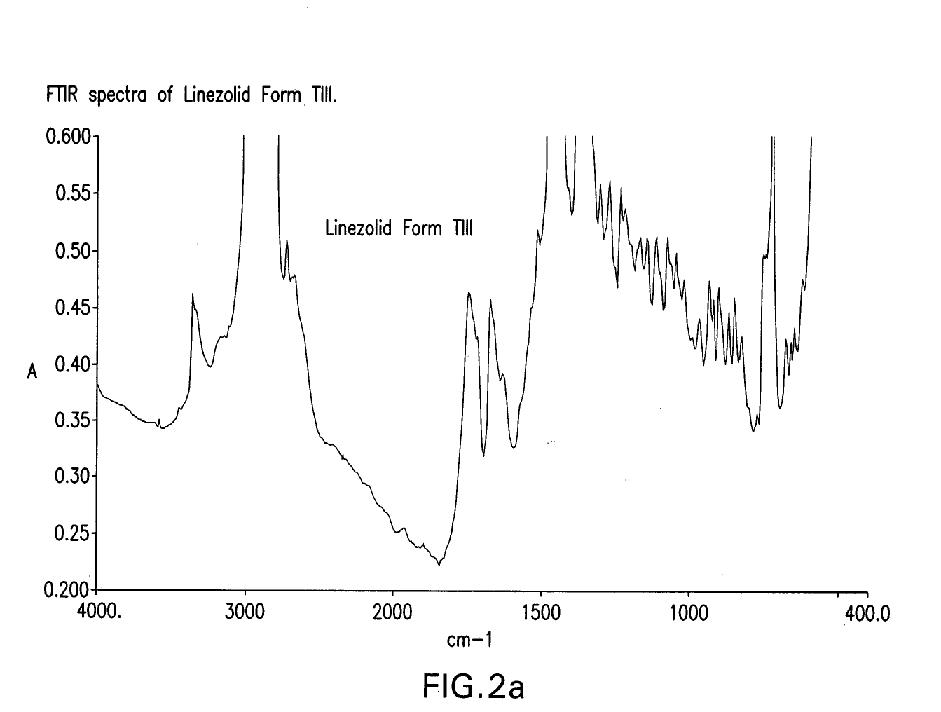

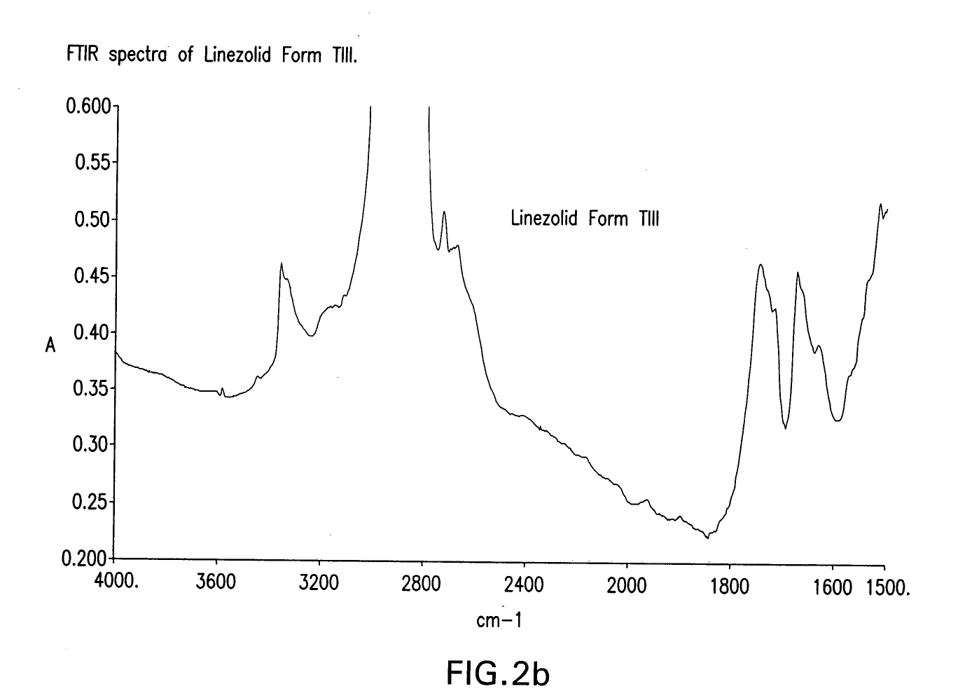

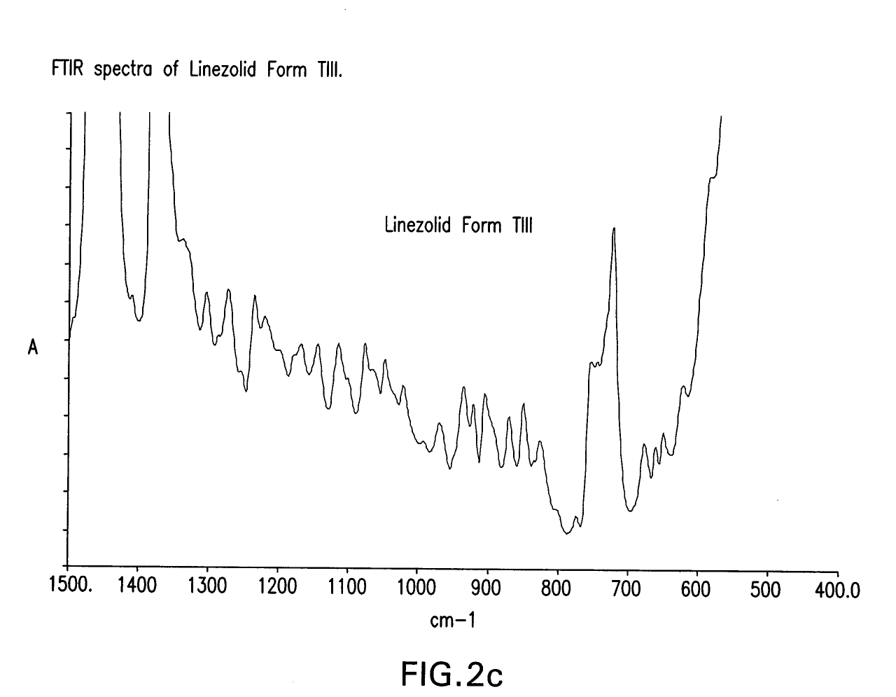

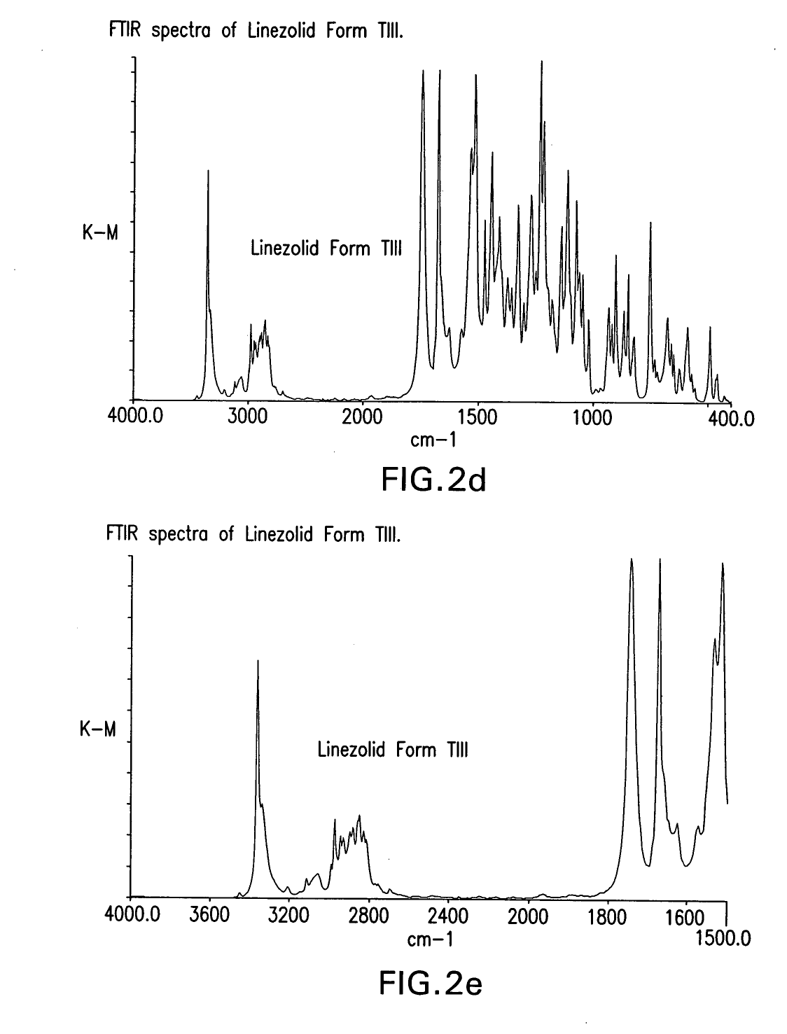

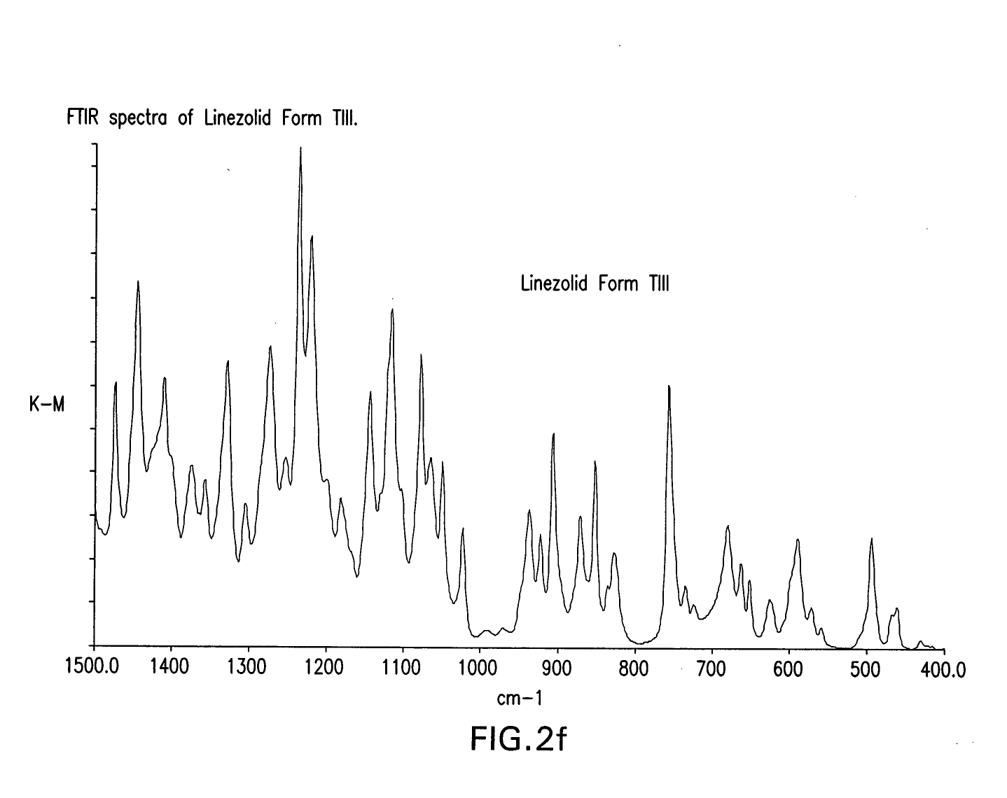

Crystalline Linezolid Form TIII may also be characterized by an FTIR spectrum having peaks specified in tables 2a and 2b. Crystalline Linezolid Form TIII may also be characterized by an FTIR spectrum substantially as depicted in FIG. 2a-2c or in FIG. 2d-2f.

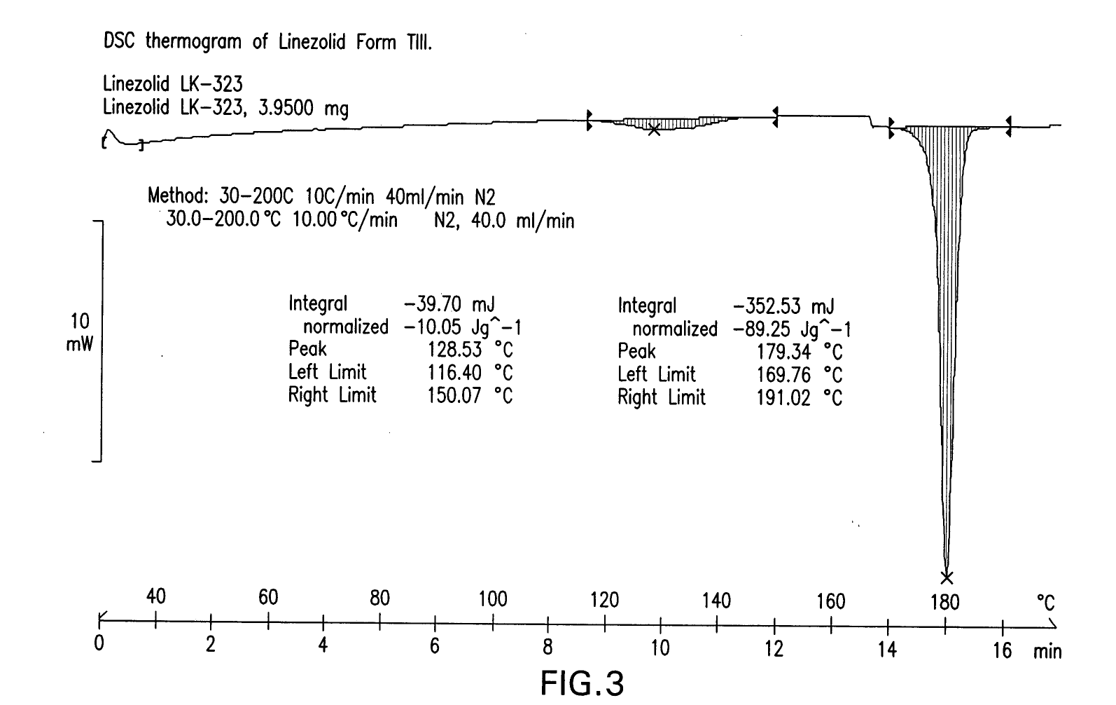

Crystalline Linezolid Form TIII may also be characterized by a differential scanning calorimetry (DSC) thermogram having a broad endothermic peak around 130° C., followed by a very sharp endothermic peak at around 180° C., which corresponds to the final melting of Linezolid (substantially as depicted in FIG. 3).

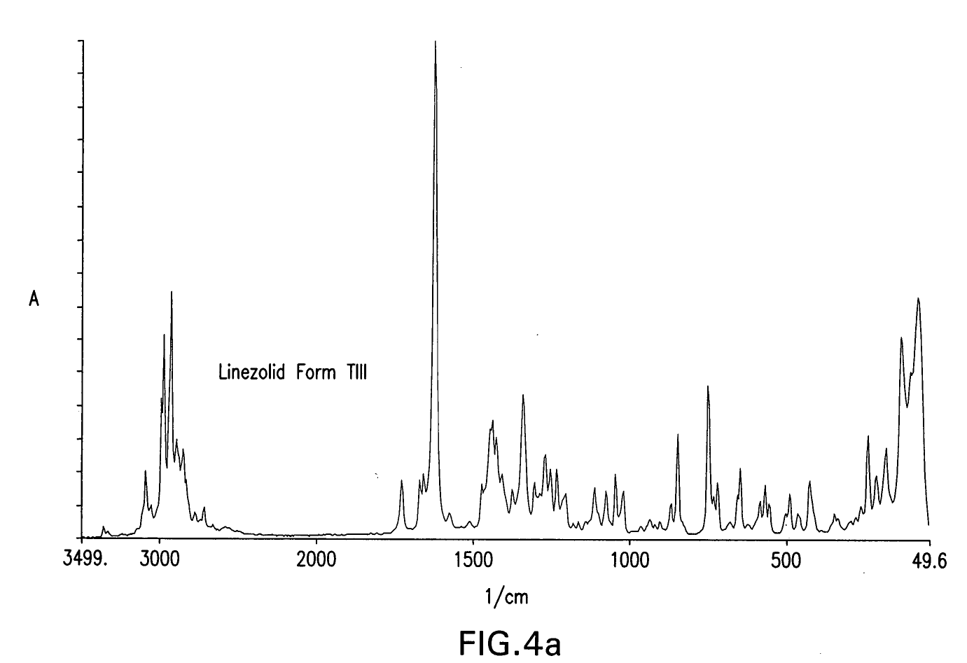

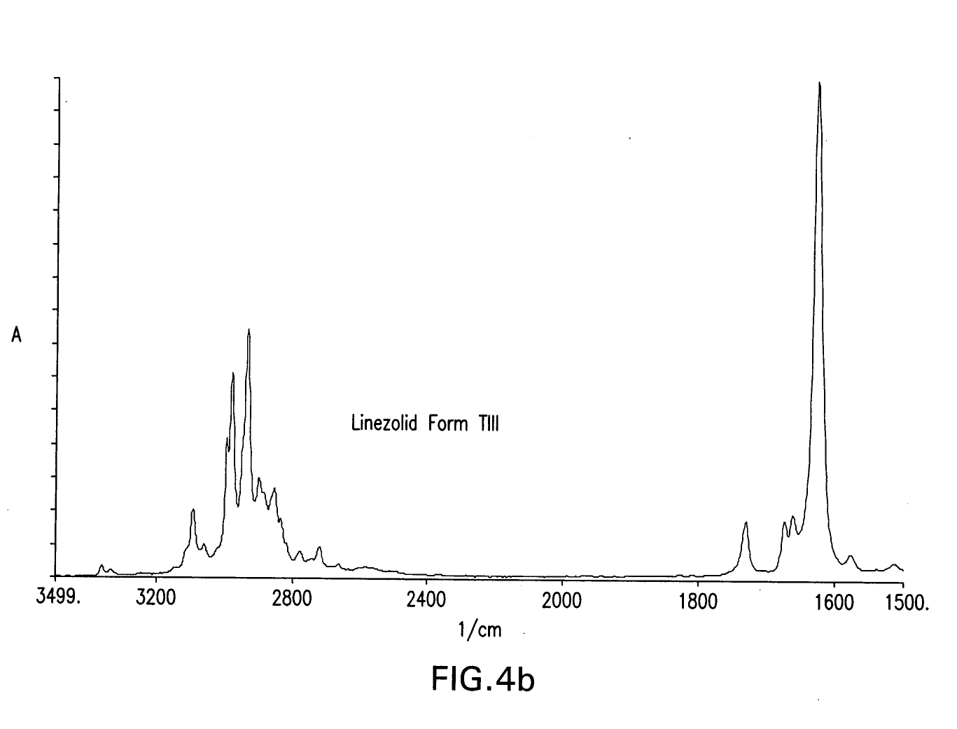

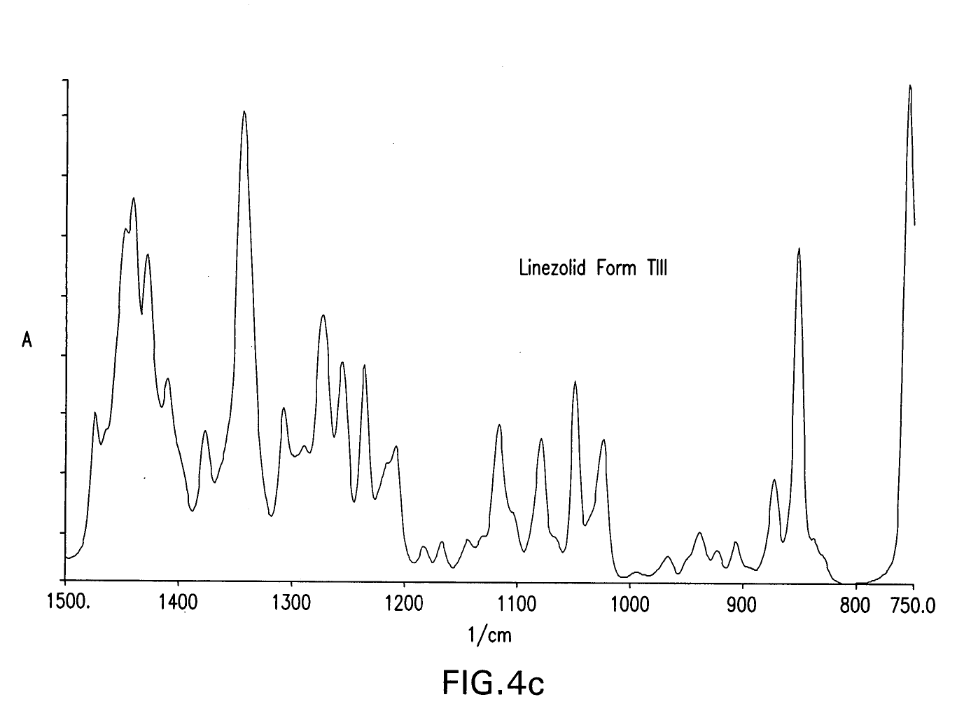

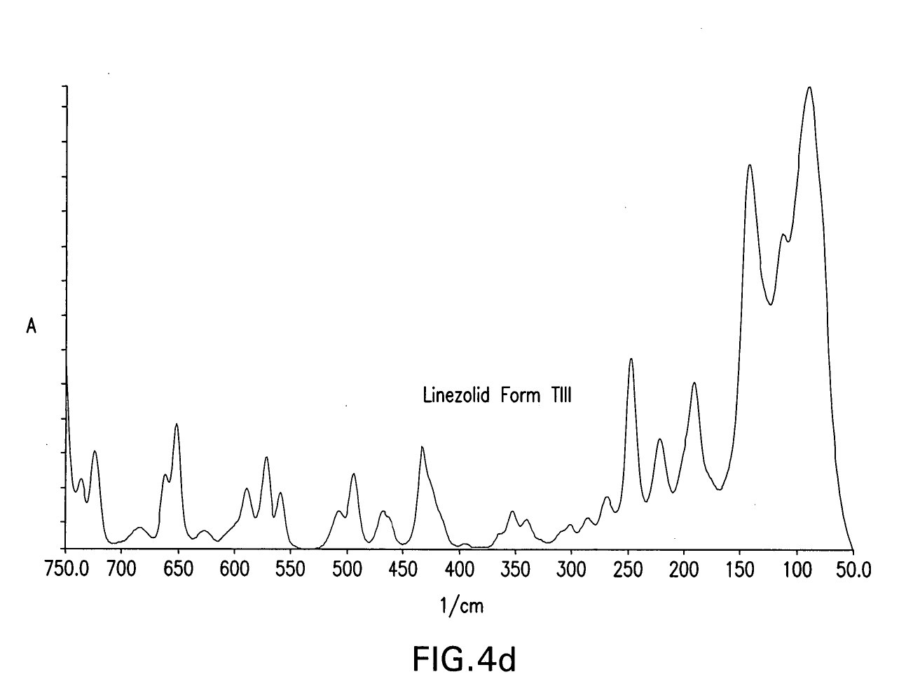

Crystalline Linezolid Form TIII may be further characterized by an FTRaman spectrum with a characteristic peak at about 724cm⁻¹. Crystalline Linezolid Form TIII may be further characterized by an FTRaman spectrum substantially as depicted in FIG. 4a-4d.

The invention encompasses crystalline Linezolid Form TIII, which has about 0.1% water by weight. Form TIII may also be characterized by a weight loss measured by thermal gravimetric analysis (TGA) of about 0.1% by weight. In this embodiment, Form TIII is anhydrous. Thus, the present invention includes crystalline Linezolid Form TIII which has about 0.1% or less water by weight.

Crystalline linezolid Form TIII is thermally stable, and does not transform into other crystalline forms upon heating at a temperature of about 60° C. to about 130° C. (see Table 4 below).

Crystalline linezolid Form TIII is further characterized by crystals having a plate shape, substantially as depicted in FIG. 30.

The present invention also provides a process for preparing crystalline Linezolid Form TIII comprising the steps of:
a) dissolving Linezolid in a polar organic solvent to obtain a solution;
b) washing the solution with water to form a two phase solution of the organic solvent and water;
c) separating the phases;
d) removing the organic solvent from the separated organic phase; and
e) recovering crystalline Linezolid Form TIII.

In the process for preparing crystalline Linezolid Form TIII, the polar organic solvent may be immiscible with water, and may be dichloromethane. The polar organic solvent may also be a mixture, e.g., methanol and ethyl acetate. The dissolving of step a) may be done at room temperature.

The recovering step may comprise filtering and drying of the crystals of Linezolid Form TIII until their weight is constant. The drying may be done in a vacuum oven at a temperature of 50° C. to 160° C.

Crystalline Linezolid Form TIII may be substantially free of Form II. Preferably, crystalline Linezolid Form TIII contains less than about 10%, preferably less than about 5%, and even more preferably less than about 1% (by weight) of Form II.

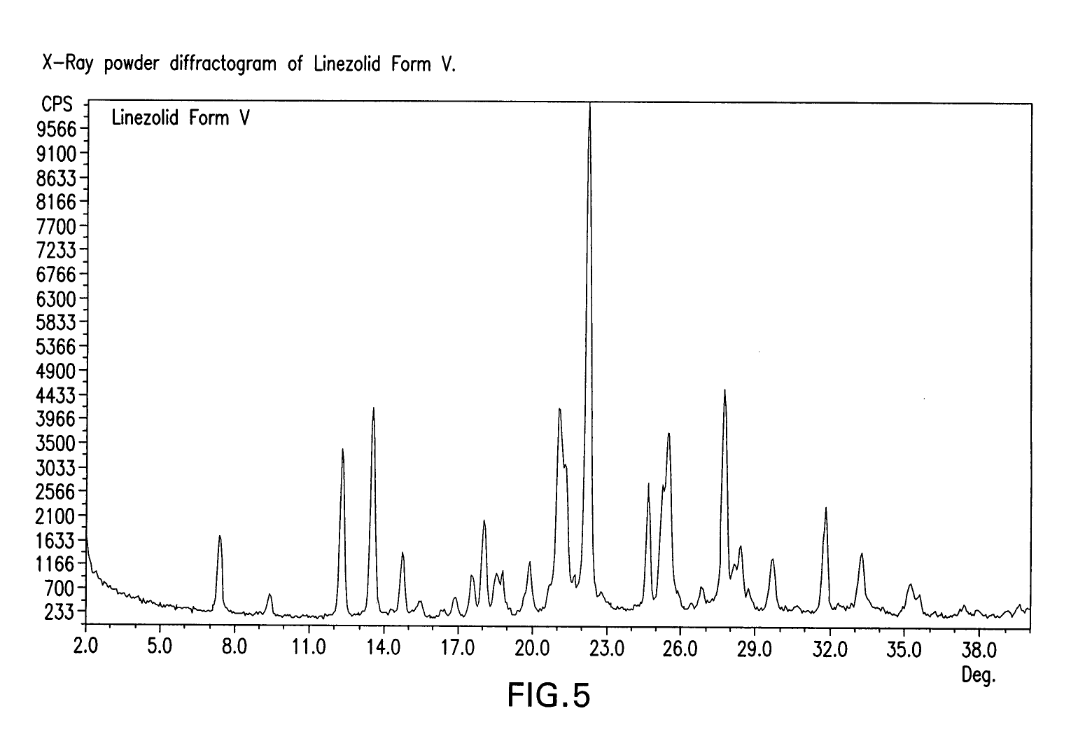

Another embodiment of the present invention is a crystalline Linezolid (denominated Form V), characterized by a PXRD pattern with peaks at about 12.3, 17.6, 22.2, 24.6, and 31.8±0.2 degrees 2 theta. Crystalline Linezolid Form V may be further characterized by PXRD peaks at about 7.5, 13.5, 21.1, 25.5, and 27.8±0.2 degrees 2 theta. Crystalline Linezolid Form V may be further characterized by PXRD pattern substantially as depicted in FIG. 5.

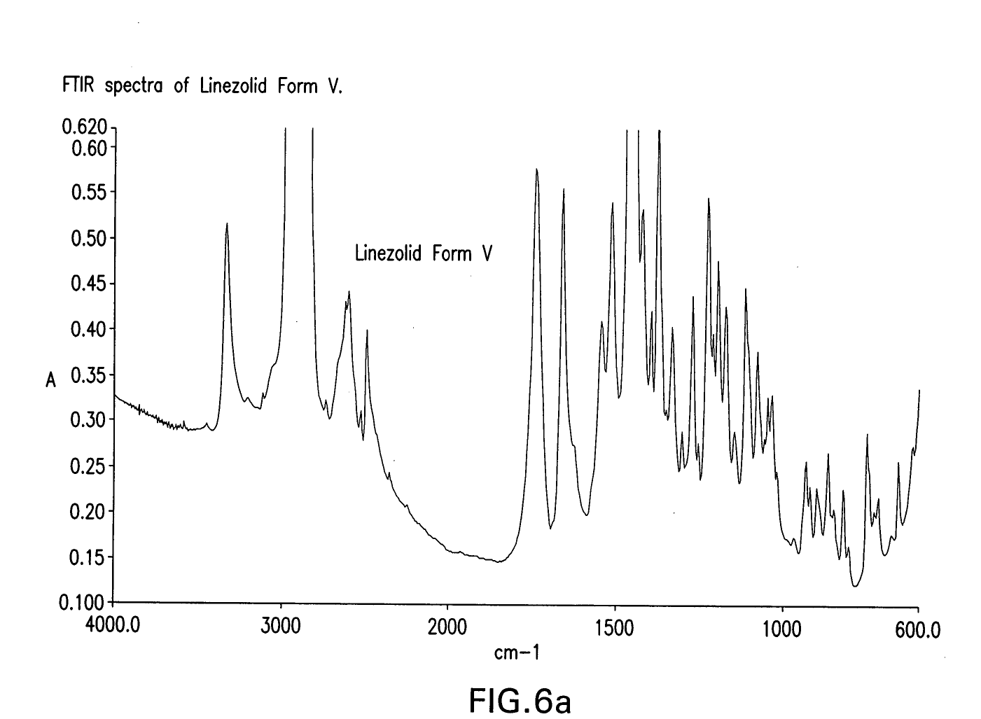

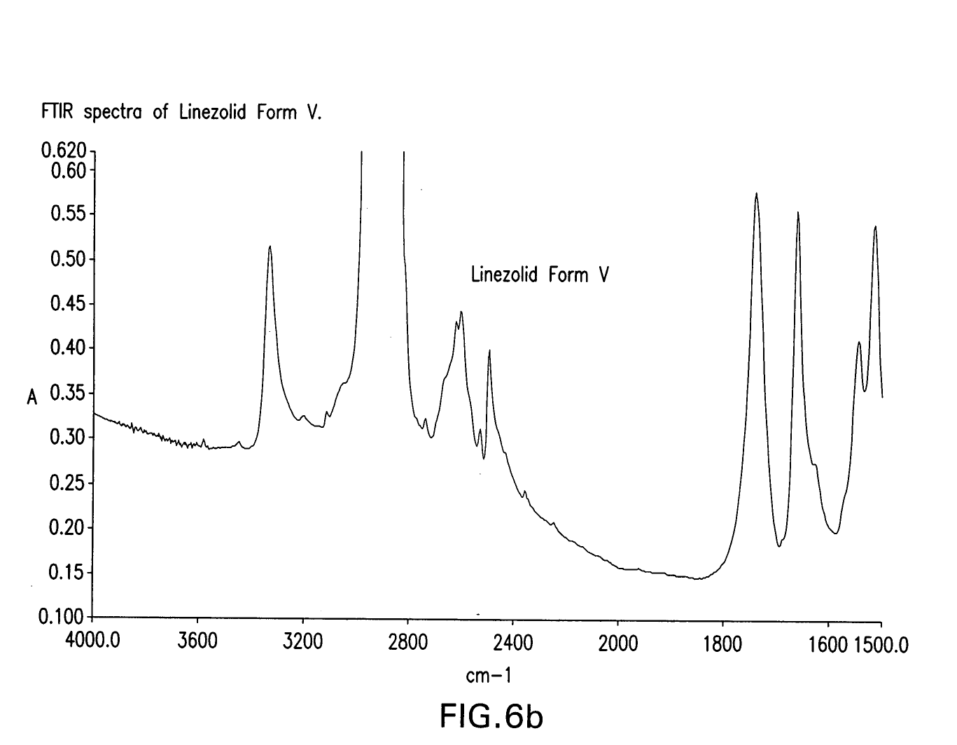

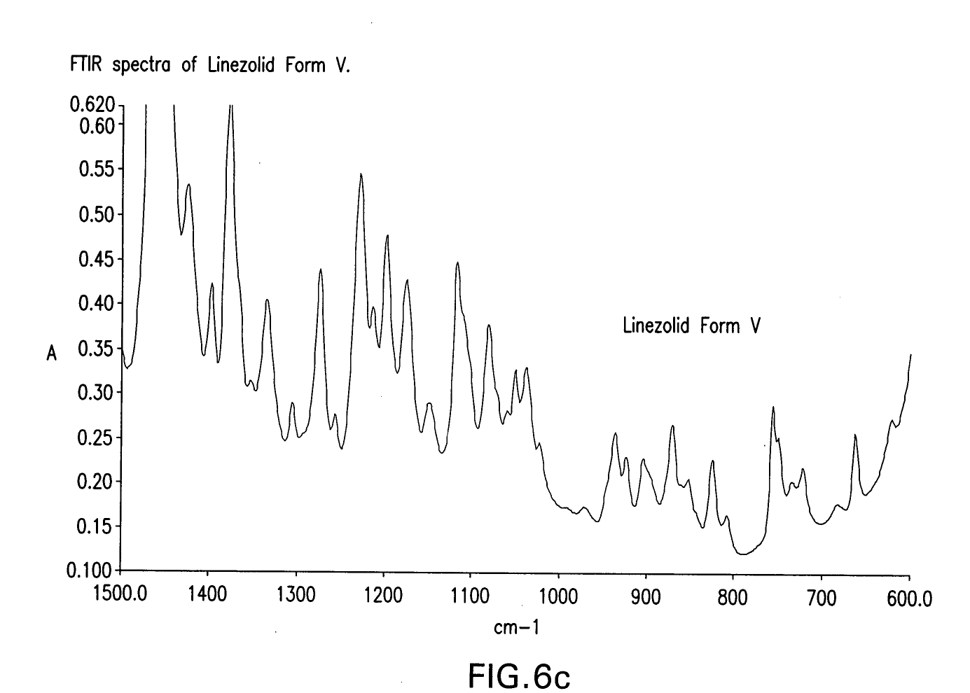

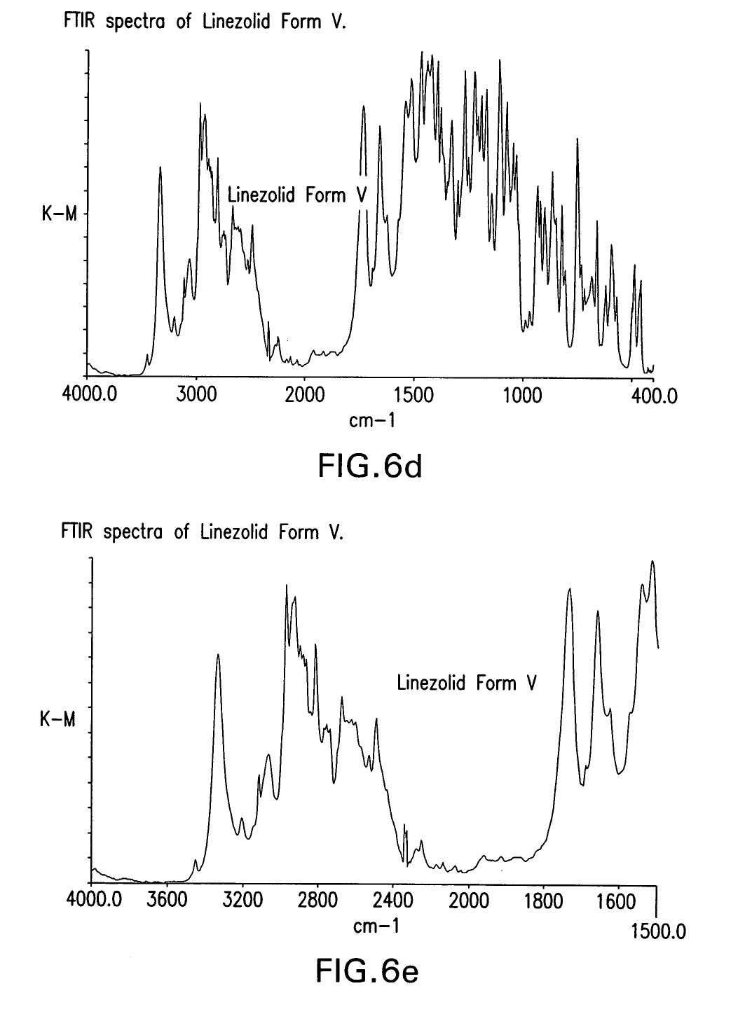

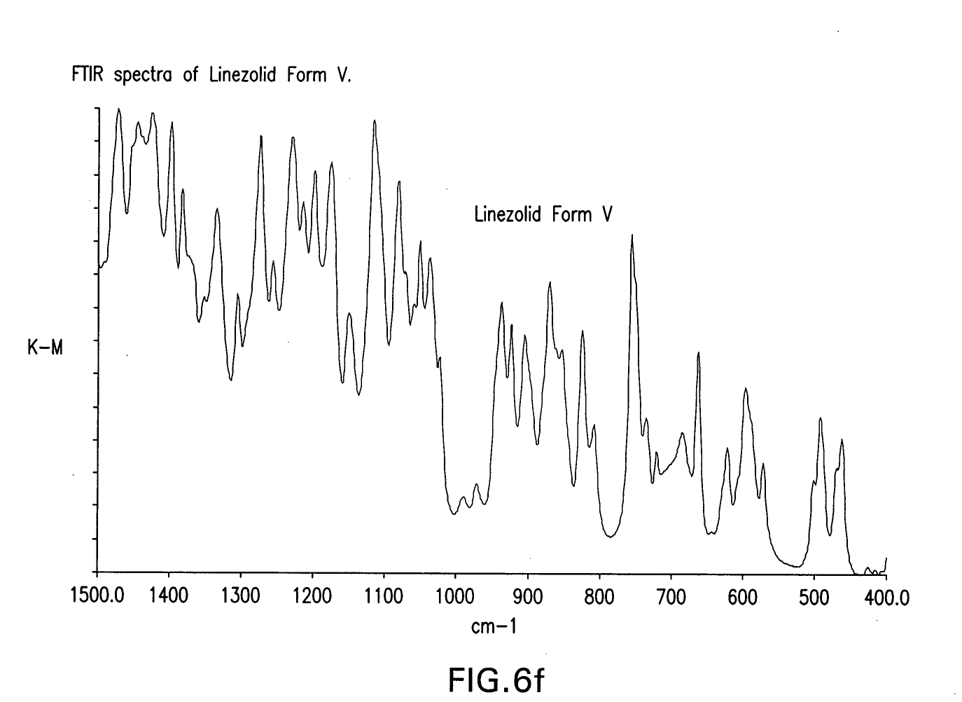

Crystalline Linezolid Form V may also be characterized by an FTIR spectrum with peaks at about 3336, 2497, 1742, 1662, 1546, 1516, 1425, 1229, and 1038 cm⁻¹. Crystalline Linezolid Form V may also be characterized by an FTIR spectrum substantially as depicted in FIG. 6a-6c or in FIG. 6d-6f.

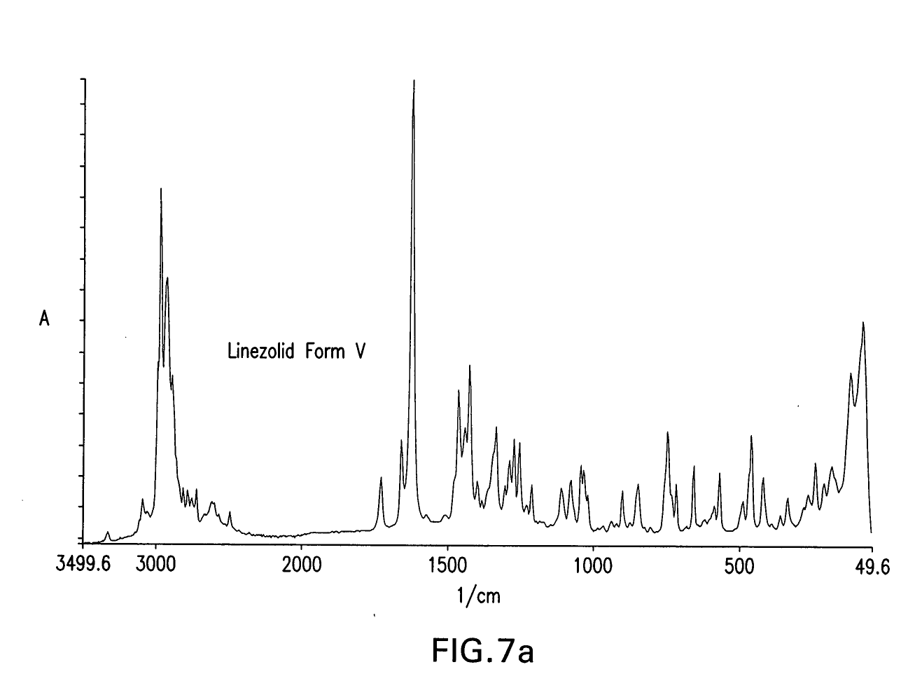

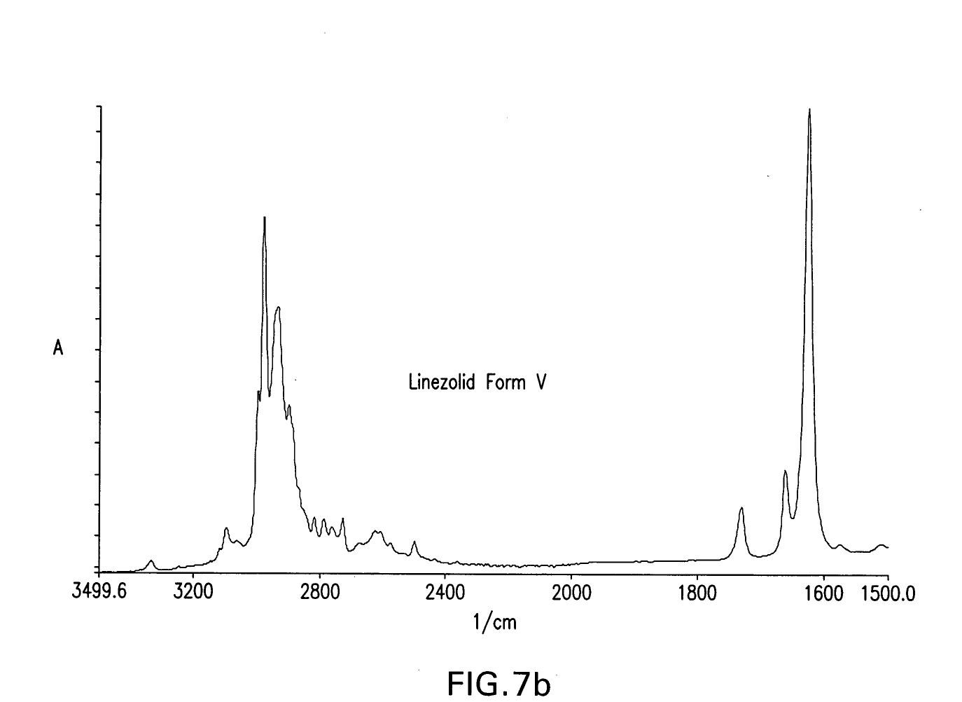

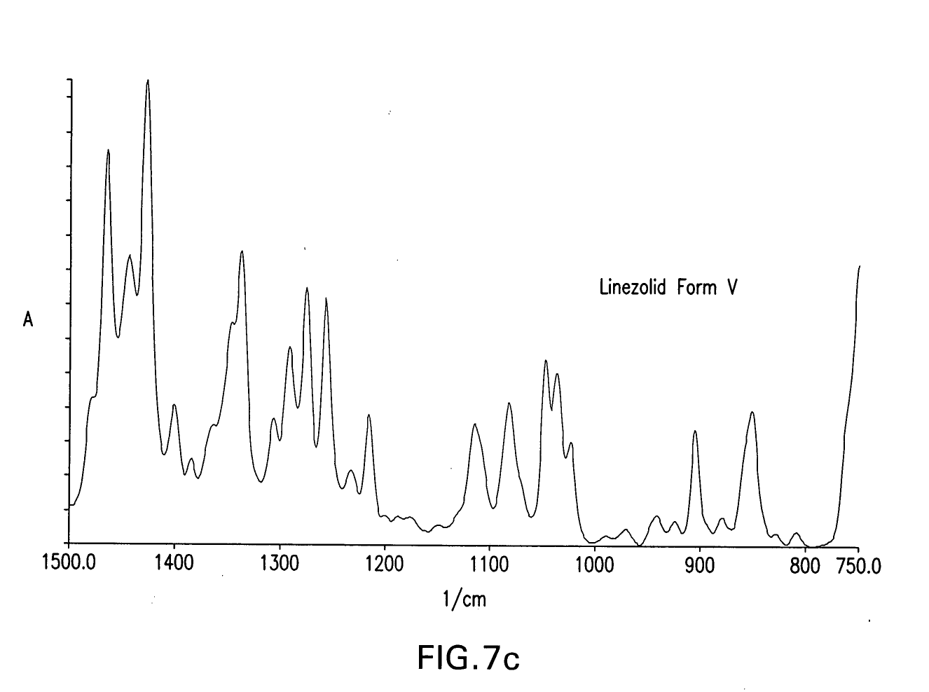

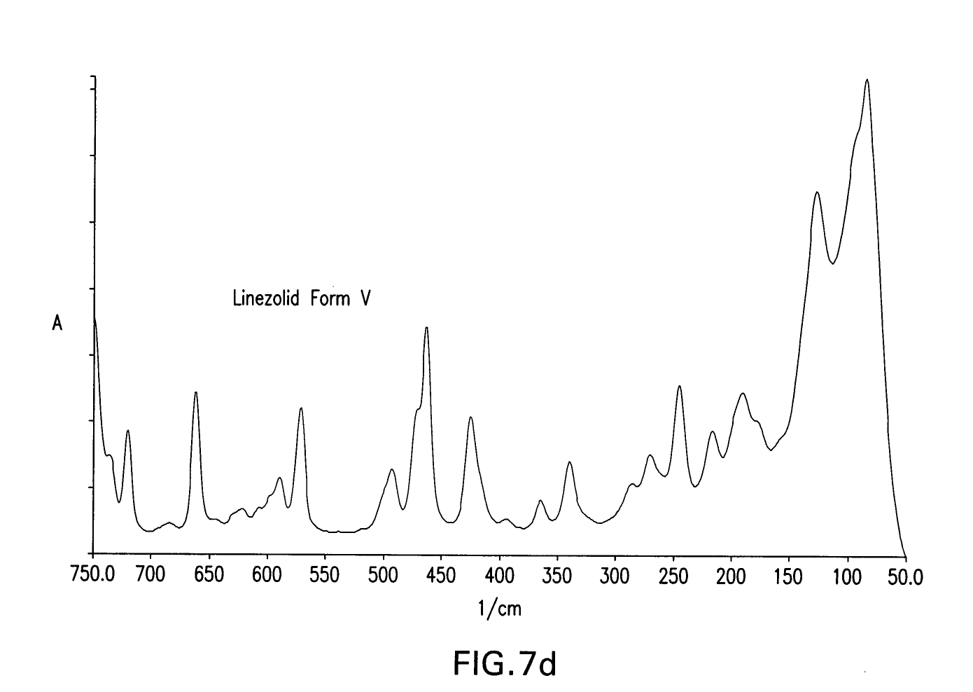

Crystalline Linezolid Form V may be further characterized by an FTRaman spectrum with peaks at about 2933, 2978, 1082 and 1036 cm⁻¹. Crystalline Linezolid Form V may be further characterized by an FTRaman spectrum with peaks at about 1660, 1428, 1465, 904, 661, 462, 424, 339 and 127 cm⁻¹. Crystalline Linezolid Form V may be further characterized by an FTRaman spectrum substantially as depicted in FIG. 7a-7d.

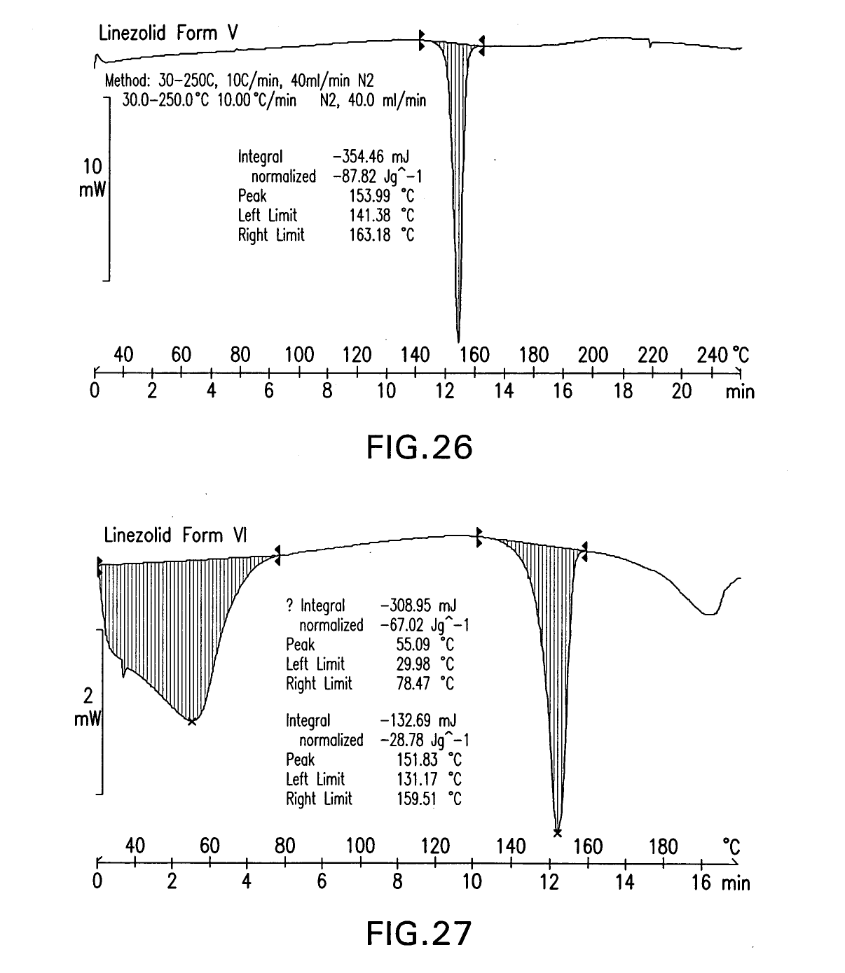

Crystalline Linezolid Form V may also be characterized by a DSC thermogram having a meltine endotherm at around 155° C., substantially as depicted in FIG. 26.

Crystalline Linezolid Form V may be substantially free of Form II. Preferably, crystalline Linezolid Form V contains less than about 10%, preferably less than about 5%, and even more preferably less than about 1% (by weight) of Form II.

The invention provides crystalline Linezolid Form V, which has about 0.1% water by weight. Form V may also be characterized by a weight loss measured by thermal gravimetric analysis (TGA) of about 0.1% by weight. This embodiment of Form V is anhydrous. Thus, the present invention includes crystalline Linezolid Form V which has about 0.1% or less water by weight.

Crystalline linezolid Form V is thermally stable, and does not transform into crystalline form IV or into the amorphous form upon heating at a temperature of about 60° C. to about 130° C. (see Table 4 below).

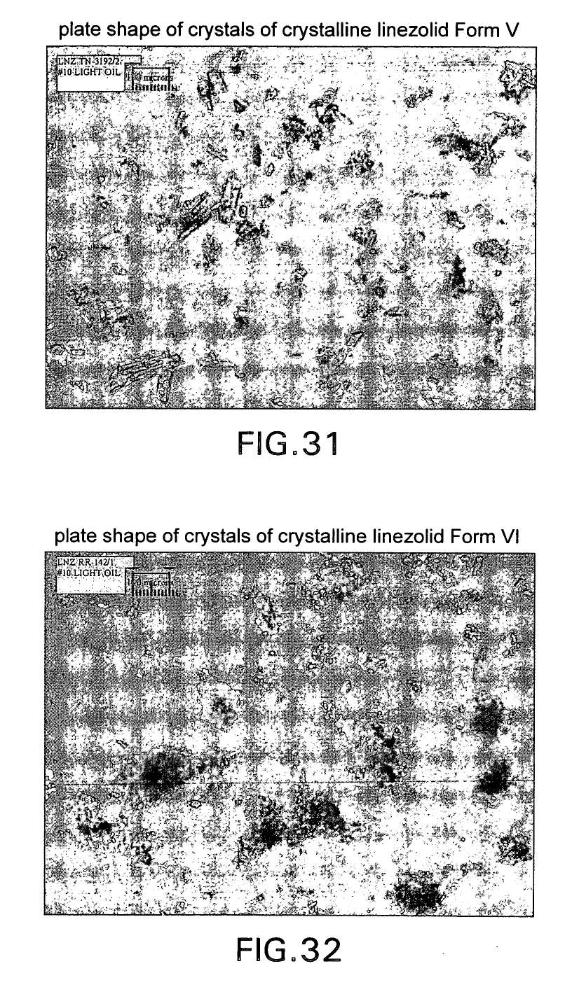

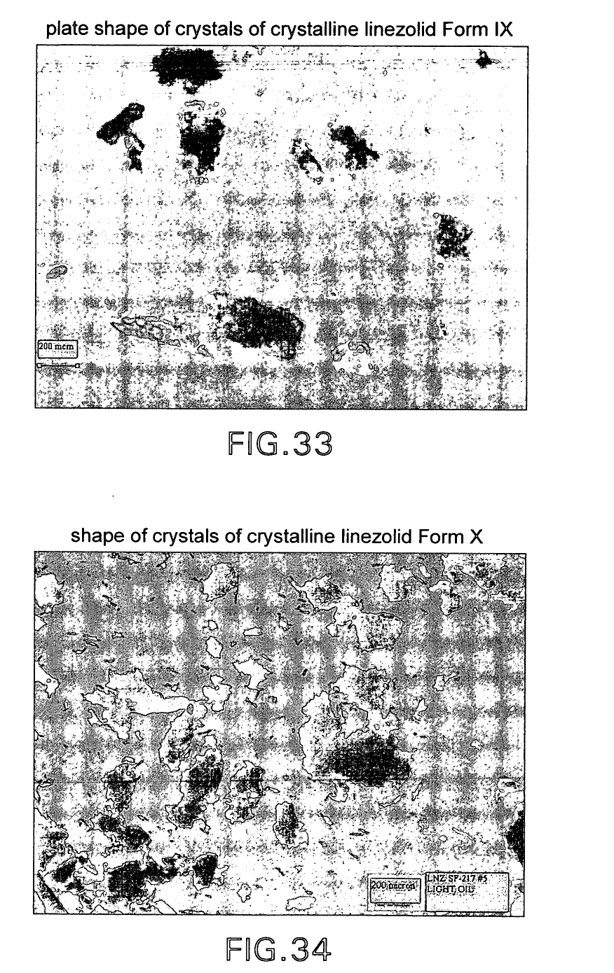

Crystalline linezolid Form V is further characterized by crystals having a plate shape, substantially as depicted in FIG. 31.

Another aspect of the present invention is a process for obtaining crystalline Linezolid Form V comprising:
a) dissolving R—N-(4-morpholinyl-3-fluorophenyl)-2-oxo-5-oxazolidinyl-methyl amine in a solution of ethyl acetate and a base;
b) cooling the solution;
c) adding an acetylating agent to the solution and maintaining the solution for at least one hour;
d) adding an anti-solvent so as to precipitate the Linezolid; and
e) recovering the precipitated Linezolid as Form V.

In particular embodiments, the dissolving occurs at room temperature, the cooling of step b) is to about 0° C., the base is triethyl amine, the acetylating agent is acetyl chloride or acetic anhydride, and/or the anti-solvent is petroleum ether.

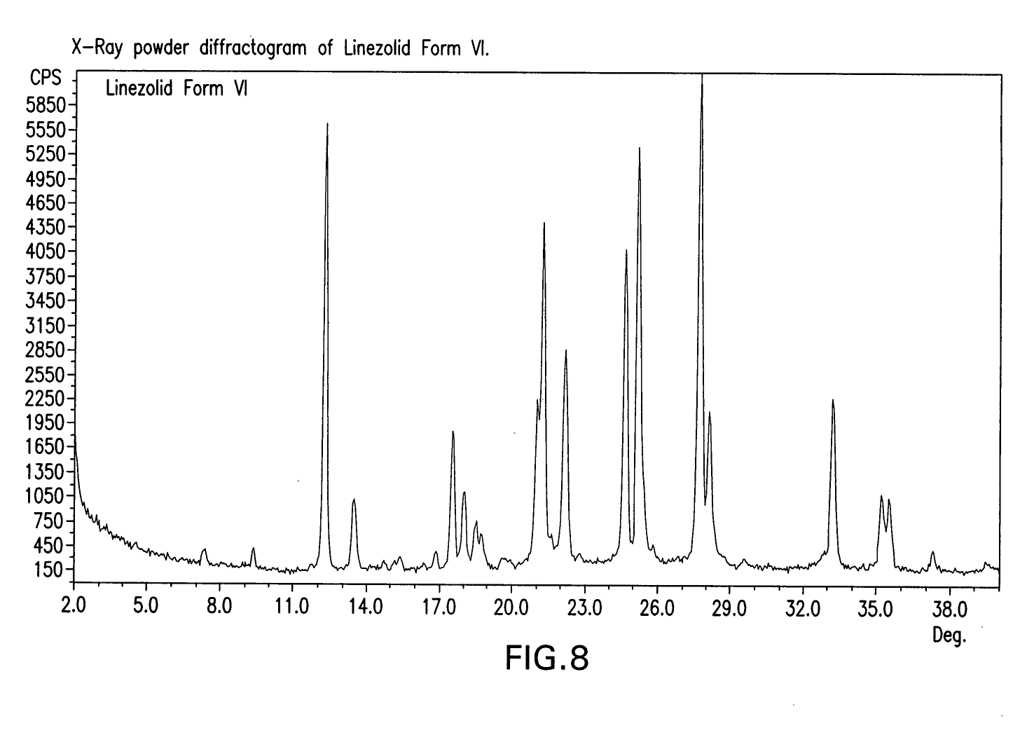

Another embodiment of the present invention is a crystalline Linezolid (denominated Form VI), characterized by a PXRD pattern with peaks at about 12.3, 21.3, 24.7, 25.2, and 27.7±0.2 degrees 2 theta. Crystalline Linezolid Form VI may be further characterized by PXRD peaks at about 17.5, 22.2, 28.1, 33.2, and 35.2±0.2 degrees 2 theta. Crystalline Linezolid Form VI may be further characterized by a PXRD pattern substantially as depicted in FIG. 8.

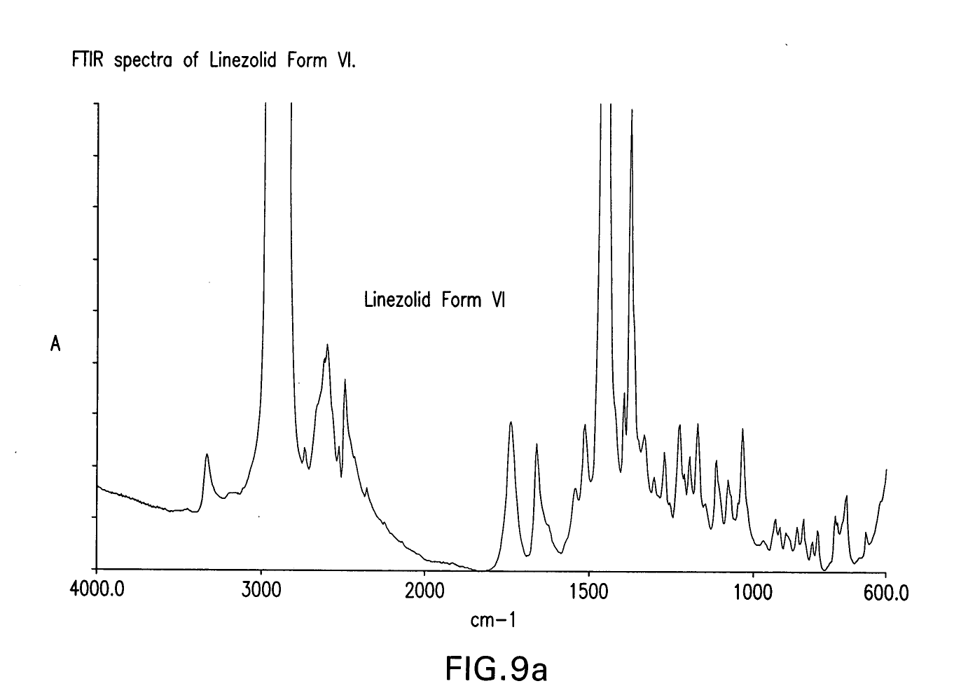

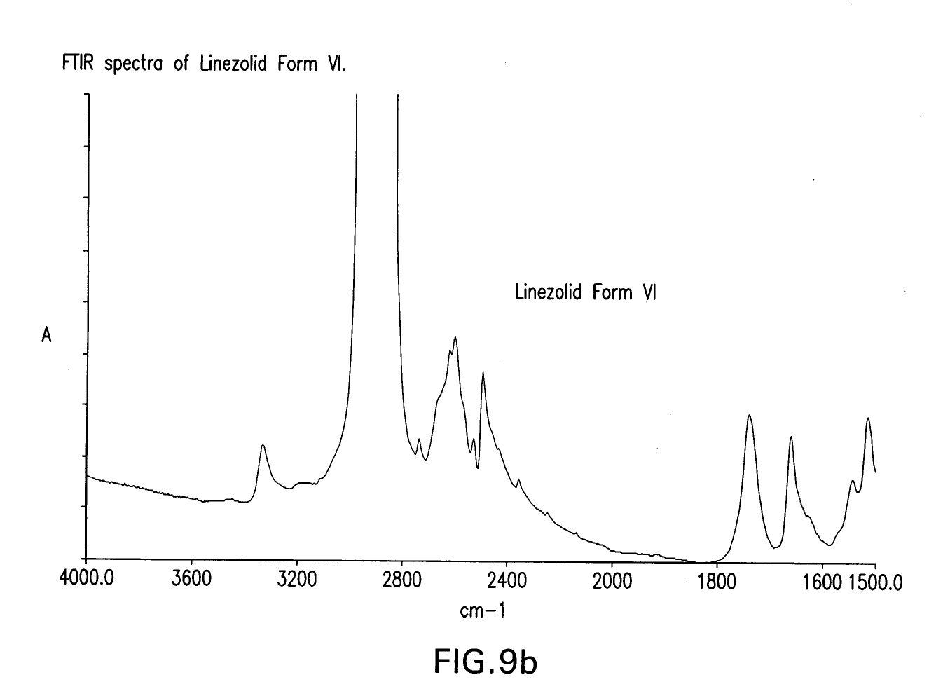

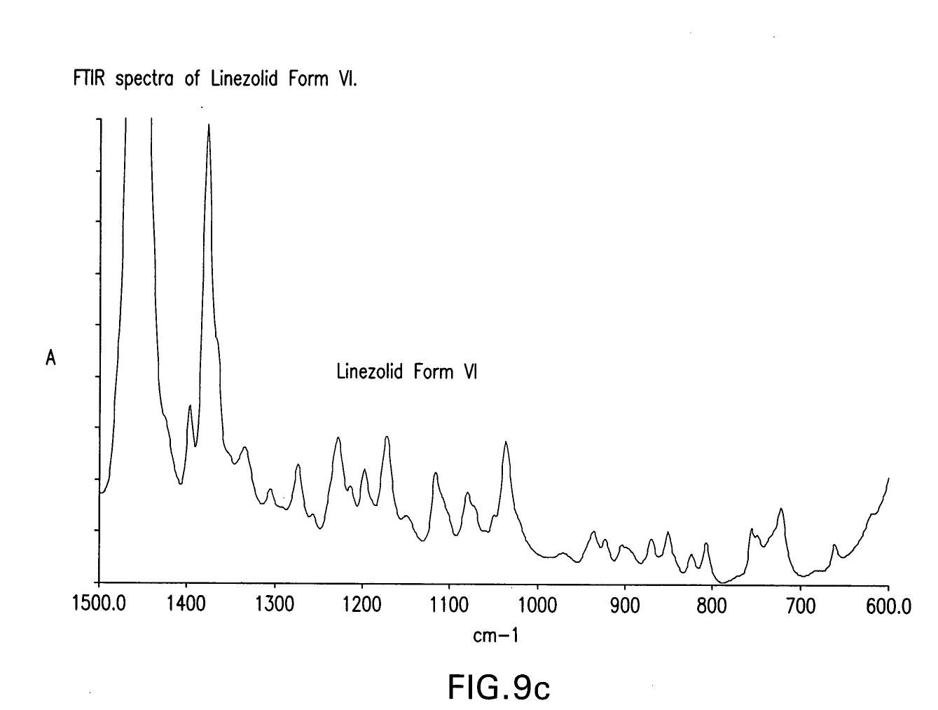

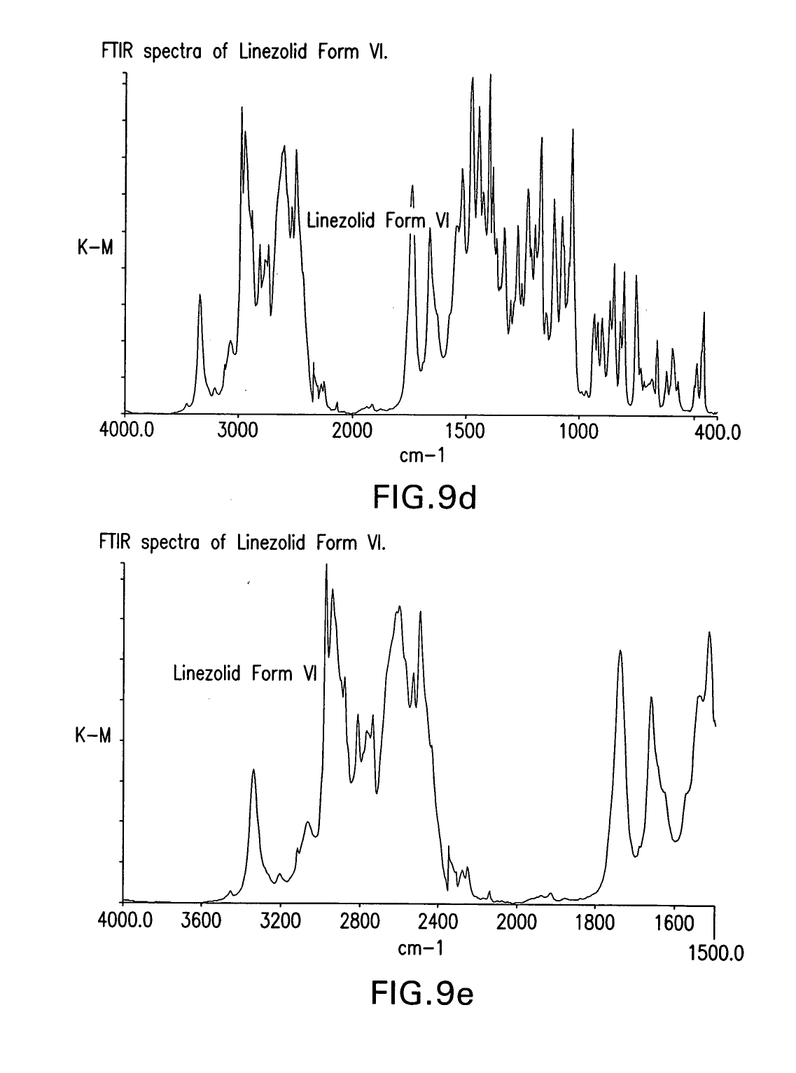

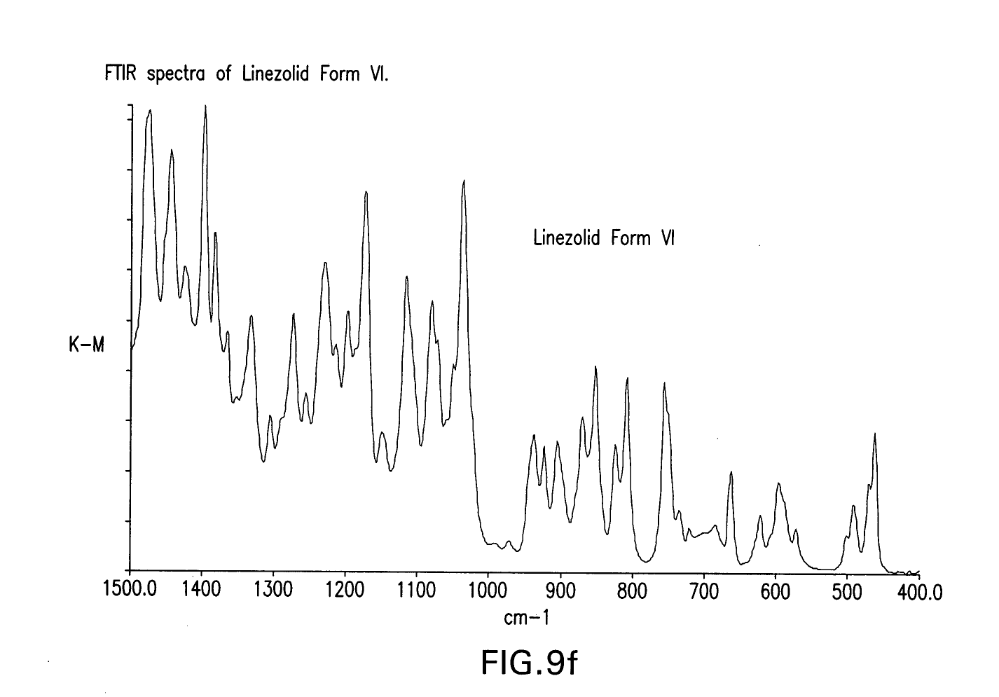

Crystalline Linezolid Form VI may also be characterized by an FTIR spectrum with peaks at about 3334, 2496, 1741, 1662, 1545, 1516, 1228, and 1036 cm⁻¹. Crystalline Linezolid Form VI may also be characterized by an FTIR spectrum substantially as depicted in FIG. 9a-9c or in FIG. 9d-9f.

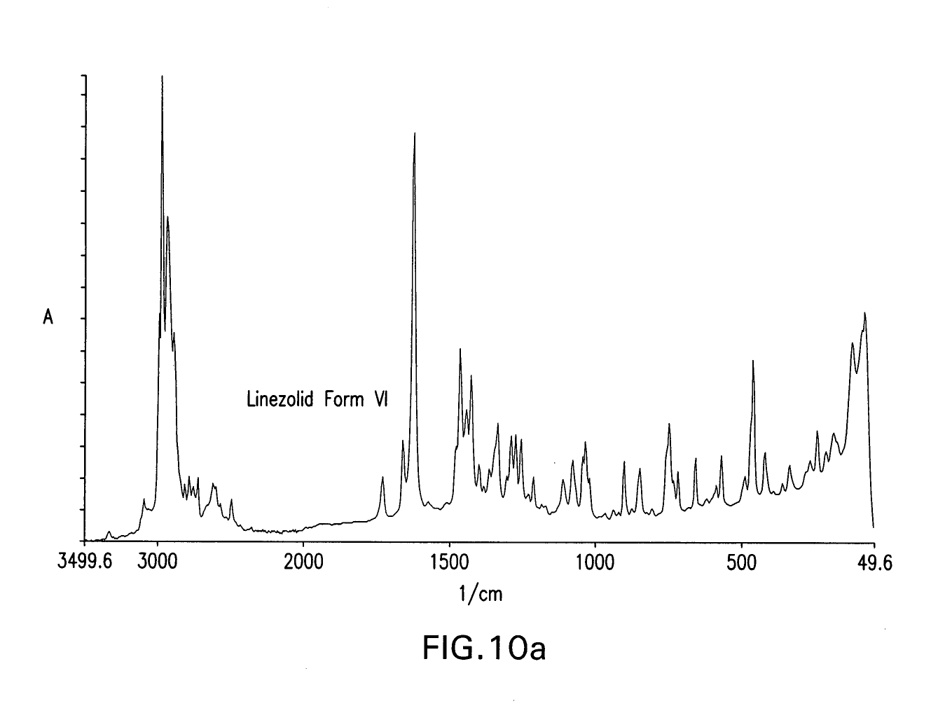

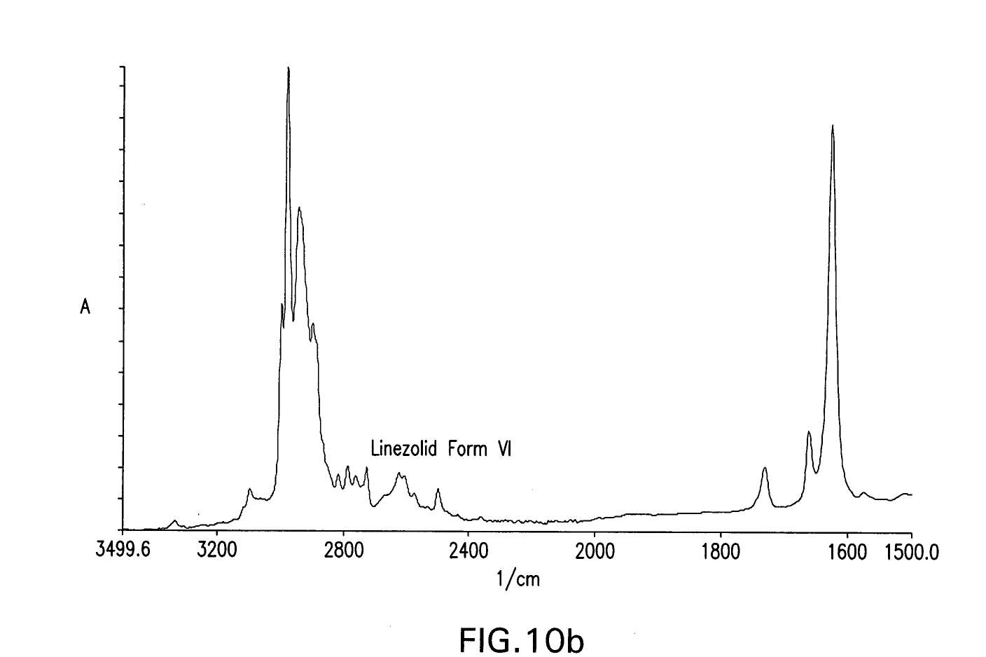

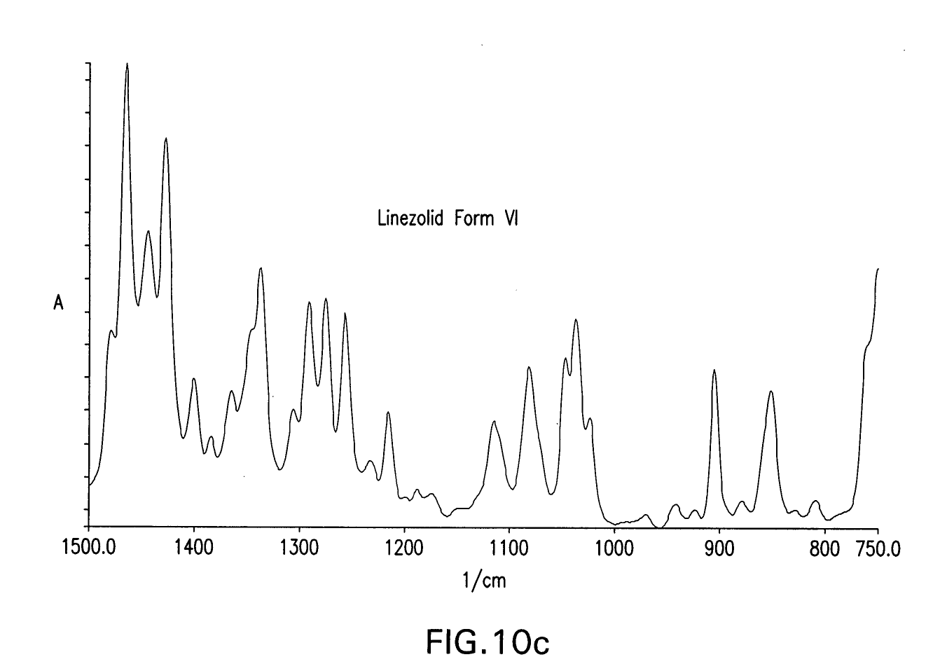

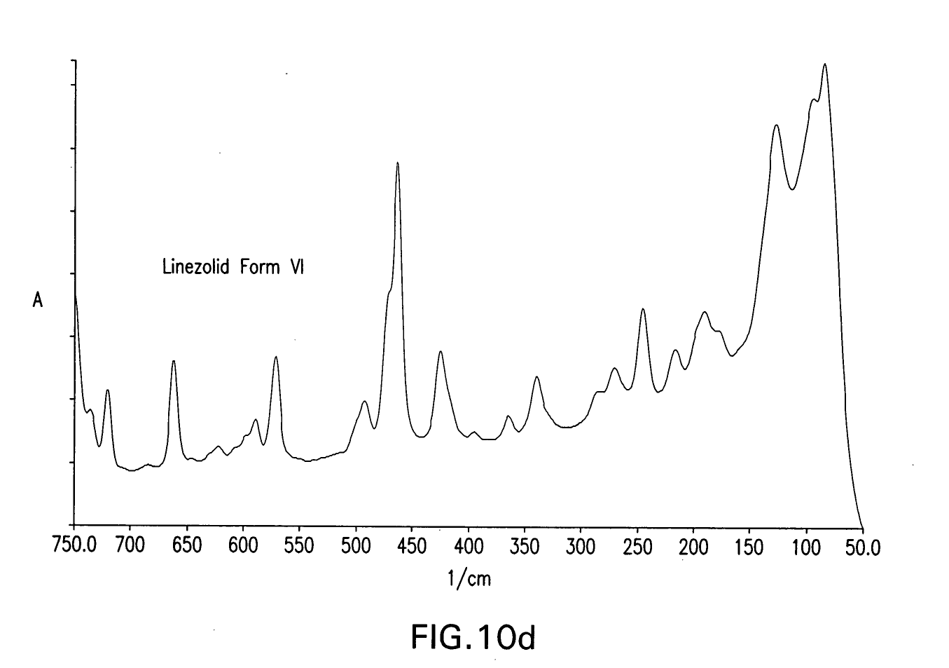

Crystalline Linezolid Form VI may be further characterized by an FTRaman spectrum with peaks at about 2996, 2942, 1037 and 463 cm⁻¹. Crystalline Linezolid Form VI may be further characterized by an additional peak in the FTRaman spectrum at about 2498 cm⁻¹. Crystalline Linezolid Form VI may be further characterized by an FTRaman spectrum substantially as depicted in FIG. 10a-10d.

Crystalline Linezolid Form VI may also be characterized by a DSC thermogram having a melting endotherm at around 155° C., substantially as depicted in FIG. 27.

Crystalline Linezolid Form VI may be substantially free of Form II. Preferably, crystalline Linezolid Form VI contains less than about 10%, preferably less than about 5%, and even more preferably less than about 1% (by weight) of Form II.

The invention provides crystalline Linezolid Form VI, which has about 0.3% water by weight. Form VI may also be characterized by a weight loss measured by thermal gravimetric analysis (TGA) of about 0.3% by weight. This embodiment of Form VI is anhydrous. Thus, the present invention includes crystalline Linezolid Form VI which has about 0.3% or less water by weight.

Crystalline linezolid Form VI is thermally stable, and does not transform into other crystalline forms upon heating at a temperature of about 60° C. to about 130° C. (see Table 4).

TABLE 4

| Initial | Heating temp. | Crystal form | Heating temp. | Crystal form |
|---|---|---|---|---|
| Form | [° C.] | after heating | [° C.] | after heating |
| TIII | 60 | TIII | 130 | TIII |
| V | 60 | V | 130 | V |
| VI | | | 130 | VI |
| X | 60 | Amorphous | 130 | IV |

The stability and hygroscopicity of linezolid Form VI was tested by storing it for 19 days at a relative humidity of between 0 and 100% at room temperature. The results are summarized in Table 5.

TABLE 5

| Relative humidity [%] | Solvent content [%] | Crystal Form |
|---|---|---|
| As is | 0.9 (water content 0.7) | VI |
| 0 | 0.4 | VI |
| 20 | 0.4 | VI |
| 40 | 0.4 | VI |
| 60 | 0.3 | VI |
| 80 | 31.7 | VI + Amorphous |
| 100 | 53.2 | Amorphous |

Form VI has proven to be stable and non-hygroscopic between 0-60% RH. However at 80-100% RH, Form VI converts to Amorphous Linezolid and on high relative humidity Form VI is hygroscopic.

Crystalline linezolid Form VI is further characterized by its crystals having a plate shape, substantially as depicted in FIG. 32.

Another aspect of the present invention is a process for obtaining crystalline Linezolid Form VI comprising:
a) dissolving R—N-(4-morpholinyl-3-fluorophenyl)-2-oxo-5-oxazolidinyl-methyl amine in a solution of ethyl acetate and a base;
b) cooling the solution;
c) adding an acetylating agent to the solution and maintainng the solution so that Linezolid Form VI precipitates; and
e) recovering the precipitated Linezolid as Form VI.

In particular embodiments, the dissolving occurs at room temperature, the cooling of step b) is to about 0° C., the base is triethyl amine, and/or the acetylating agent is acetyl chloride or acetic anhydride.

The present invention provides a crystalline racemate of linezolid.

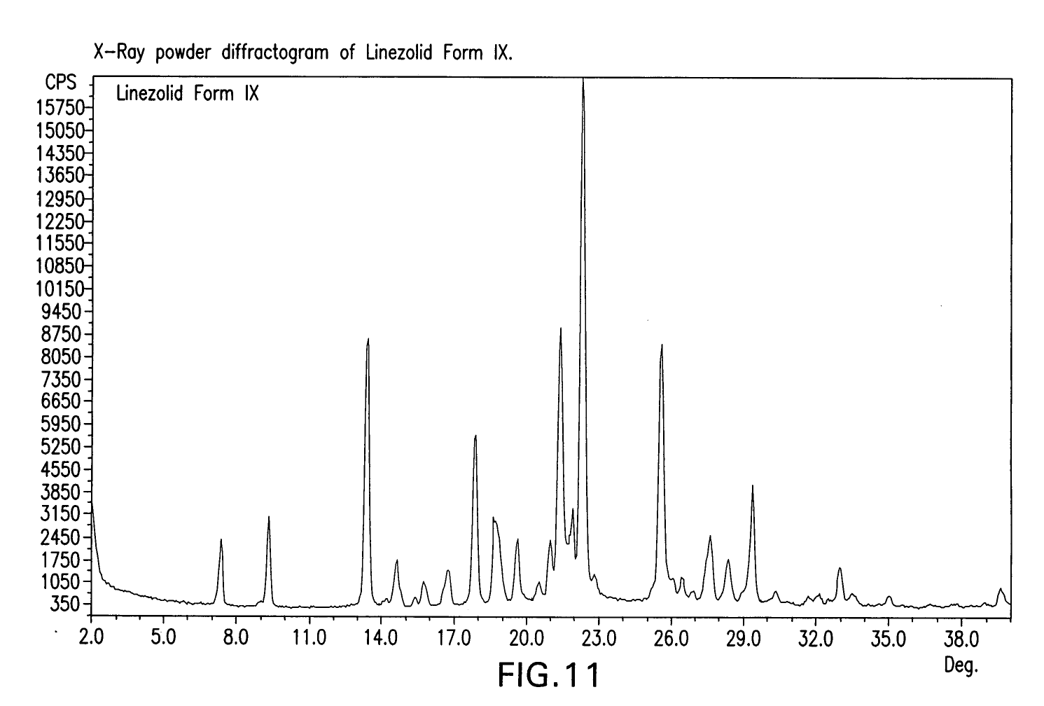

The present invention further provides a crystalline Linezolid racemate (denominated Form IX), characterized by a PXRD pattern with peaks at about 13.4, 17.9, 21.4, 22.3, and 25.6±0.2 degrees 2 theta. Crystalline Linezolid Form IX may be further characterized by PXRD peaks at about 7.3, 9.3, 18.6, and 29.3±0.2 degrees 2 theta. Crystalline Linezolid Form IX may be further characterized by a PXRD pattern substantially as depicted in FIG. 11.

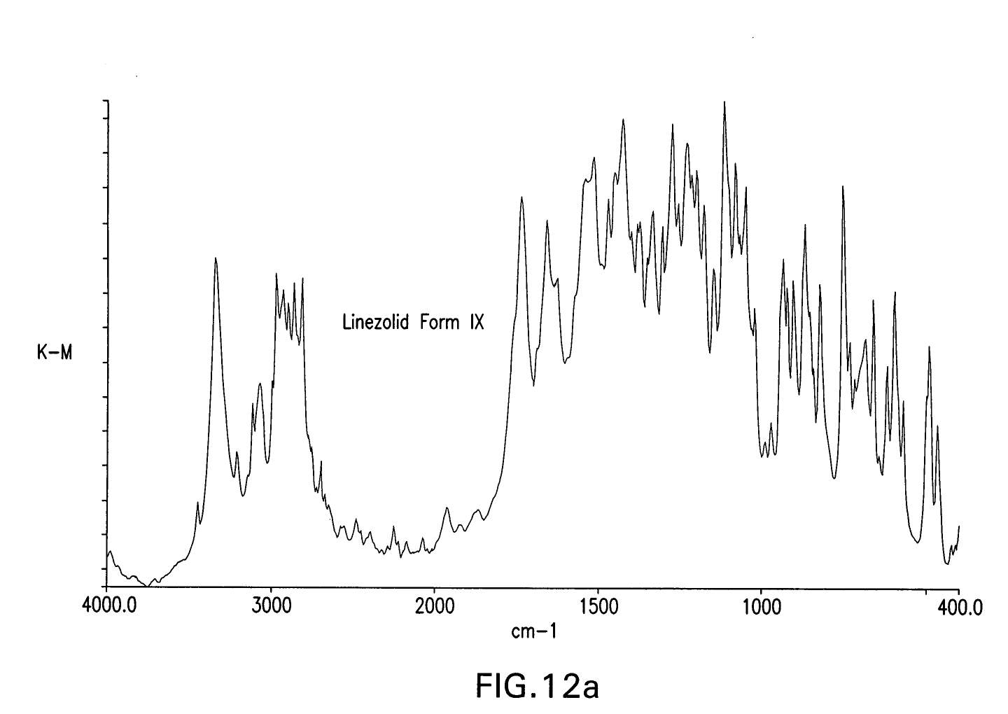

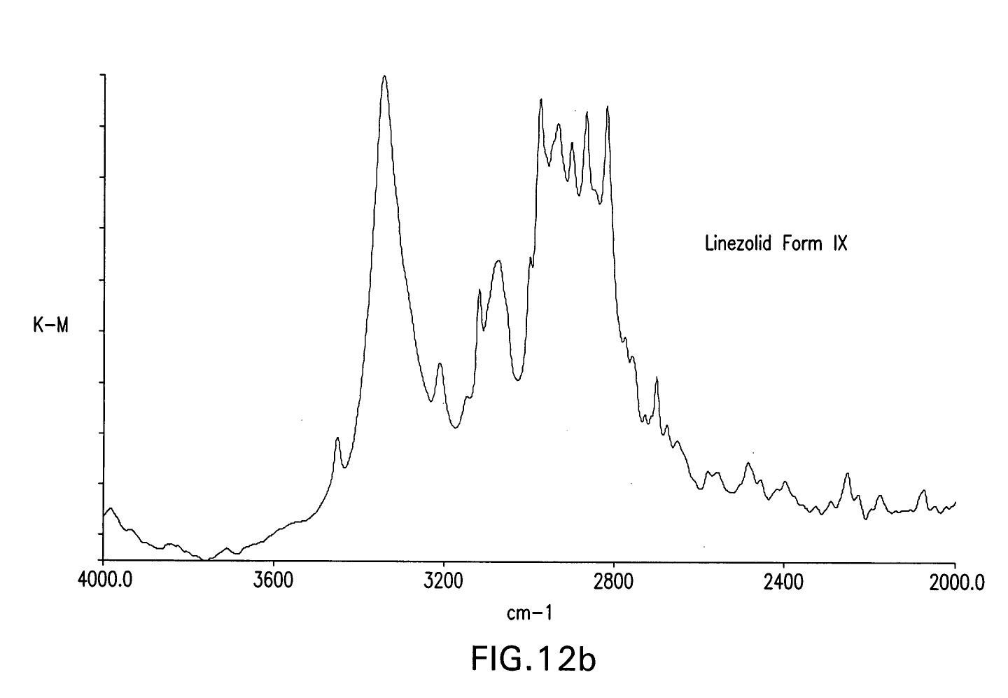

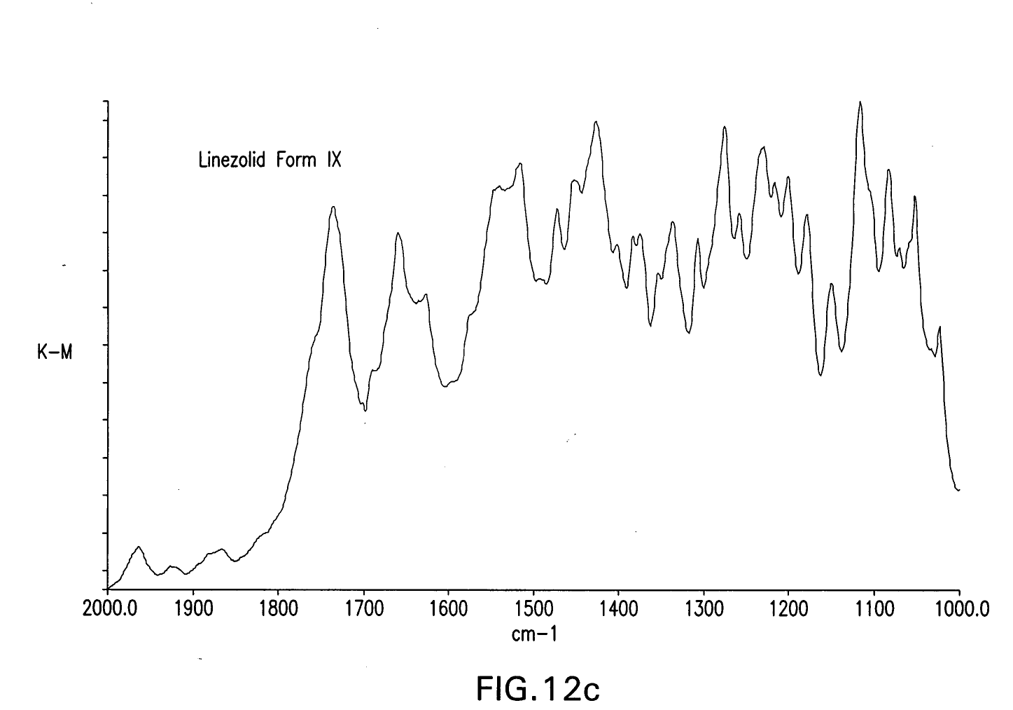

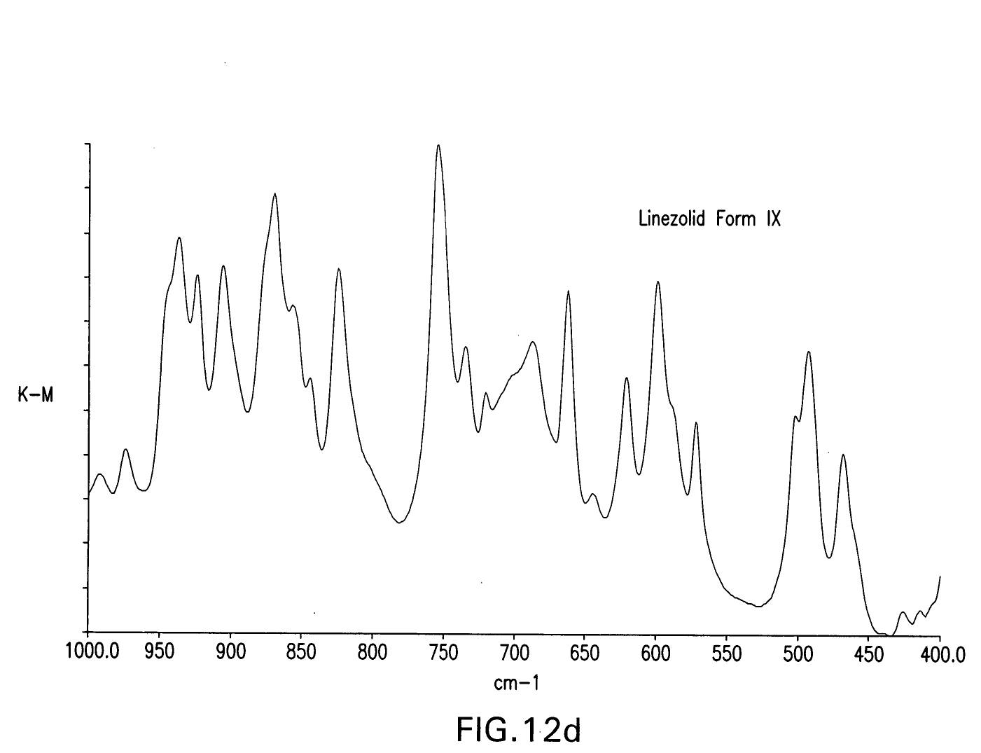

Crystalline Linezolid Form IX may be further characterized by an FTIR spectrum substantially as depicted in FIG. 12a-12d.

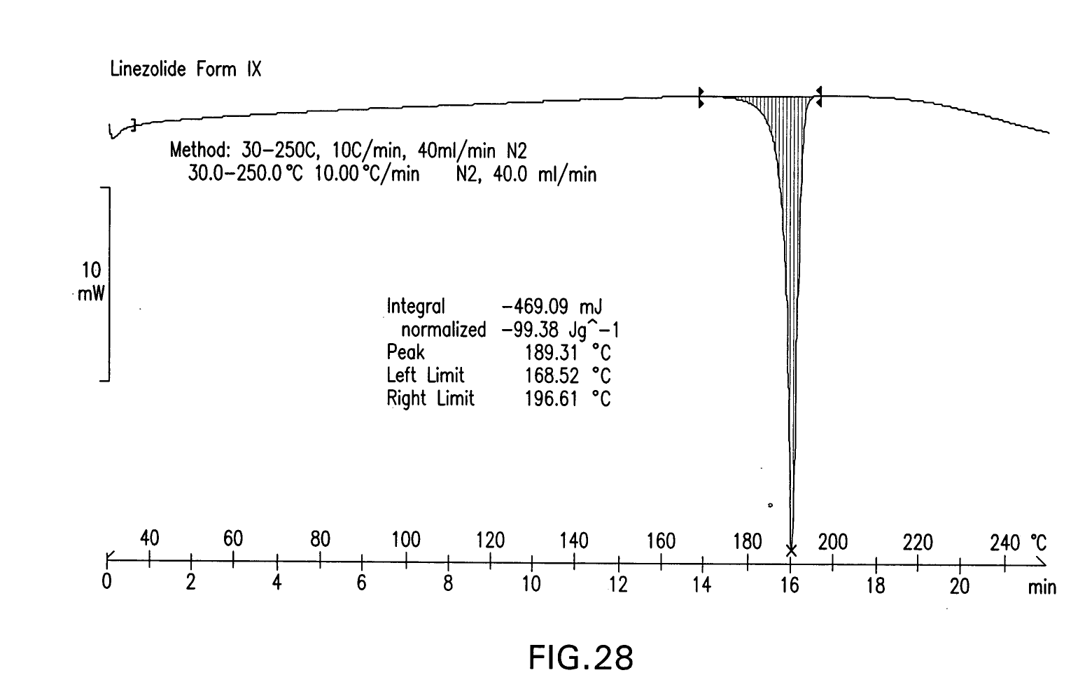

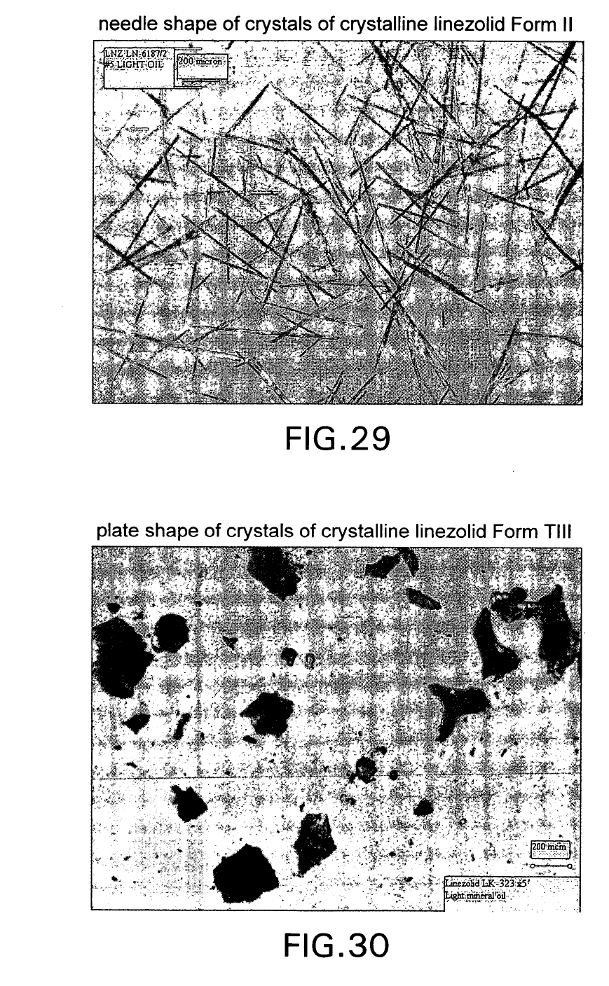

Crystalline Linezolid Form IX may also be characterized by a DSC thermogram having a meltine endotherm at around 190° C., substantially as depicted in FIG. 28.

The invention provides crystalline Linezolid Form IX, which has about 0.2% water by weight. Form IX may also be characterized by a weight loss measured by thermal gravimetric analysis (TGA) of about 0.2% by weight. This embodiment of Form IX is anhydrous. Thus, the present invention includes crystalline Linezolid Form IX which has about 0.2% or less water by weight.

Crystalline Linezolid Form IX may be substantially free of Form II. Preferably, crystalline Linezolid Form IX contains less than about 10%, preferably less than about 5%, and even more preferably less than about 1% (by weight) of Form II.

Crystalline linezolid Form IX is further characterized by its crystals having a plate shape, substantially as depicted in FIG. 33.

Another aspect of the present invention is a process for obtaining crystalline Linezolid Form IX comprising:
a) combining carbobenzoxy-3-fluoro-4-morpholinyl-aniline, t-Boc, methanol and THF to form a mixture;
b) adding (±)-N-[2-(acetyloxy)-3-chloropropyl]acetamide to the mixture of step a);
c) adding water, methylene chloride, and acetic acid to the reaction mixture of step b) to form a two-phase solution having an aqueous phase and an organic phase;
d) recovering and drying the organic phase;
e) slurrying the dried organic phase in hexane to form crystalline Linezolid Form IX; and
f) recovering the crystalline Linezolid Form IX.

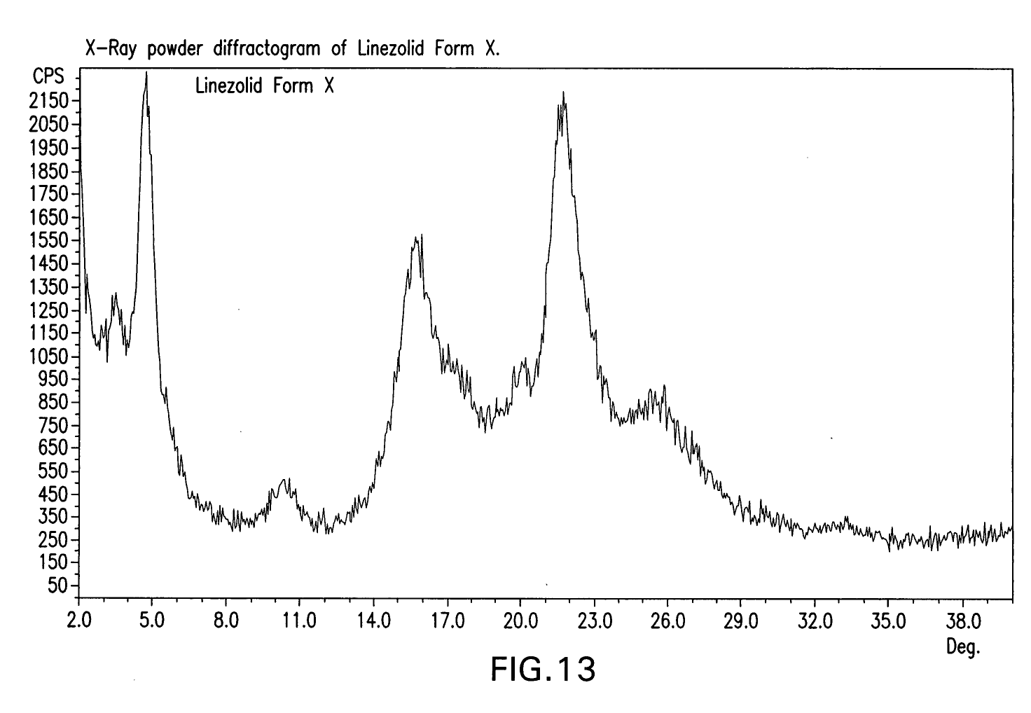

Another embodiment of the present invention is a crystalline Linezolid (denominated Form X), characterized by a PXRD pattern with peaks at about 4.7, 15.7, and 21.7±0.2 degrees 2 theta. Crystalline Linezolid Form X may be further characterized by PXRD peaks at about 3.5, 10.3, and 20.2±0.2 degrees 2 theta. Crystalline Linezolid Form X may be further characterized by a PXRD pattern substantially as depicted in FIG. 13.

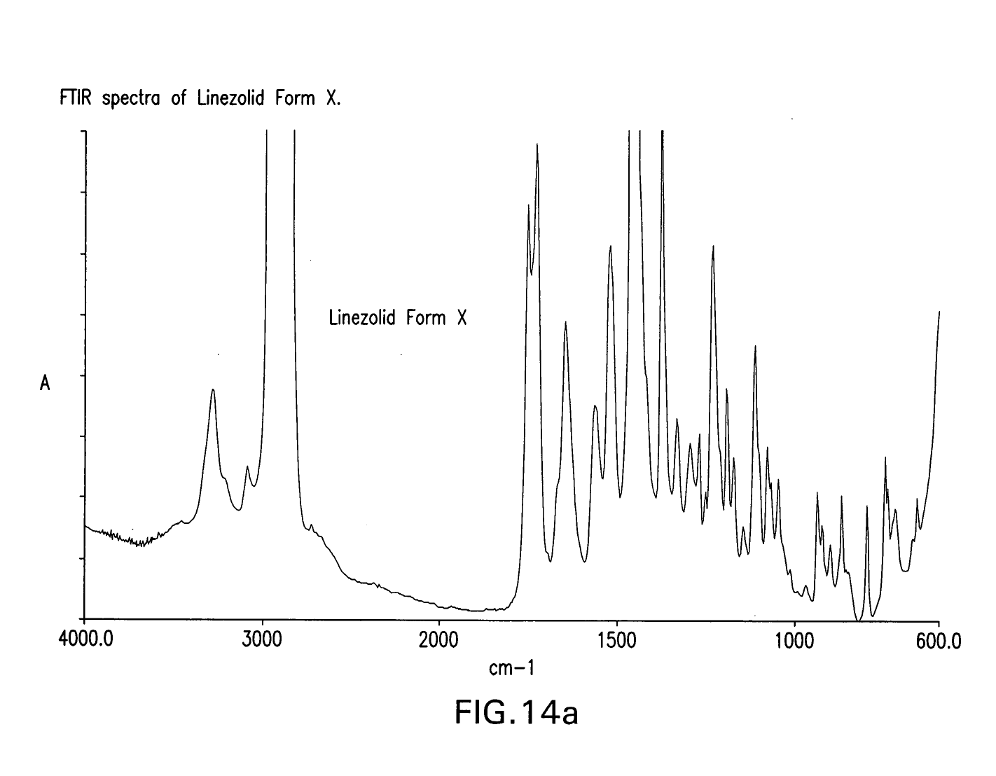

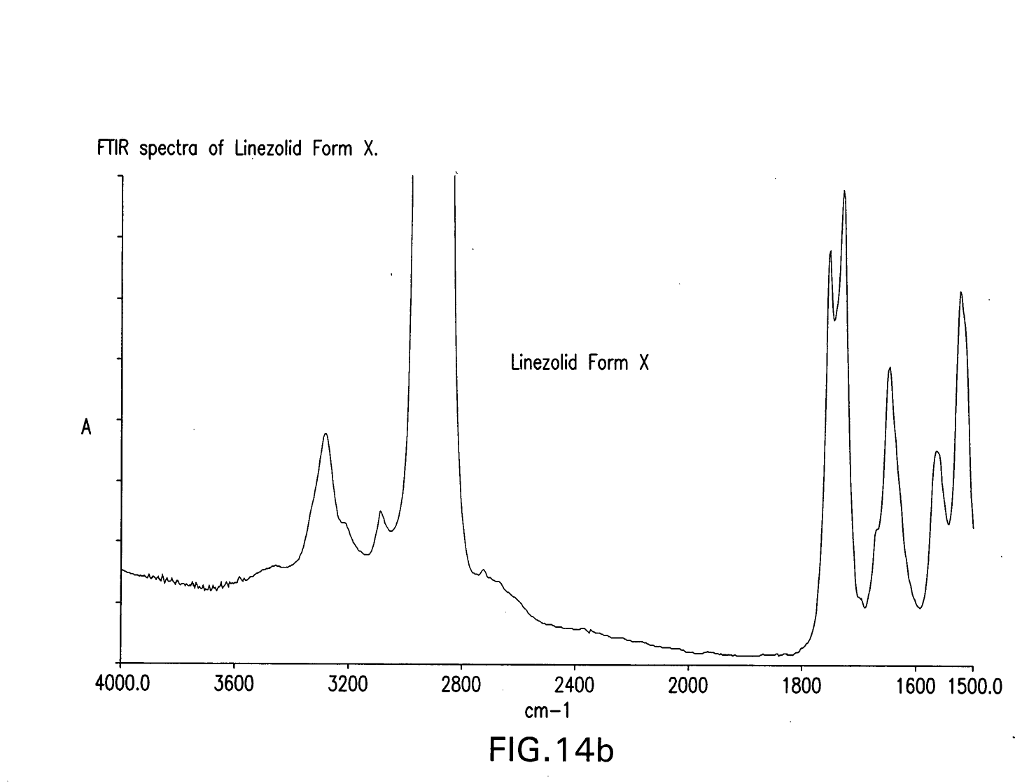

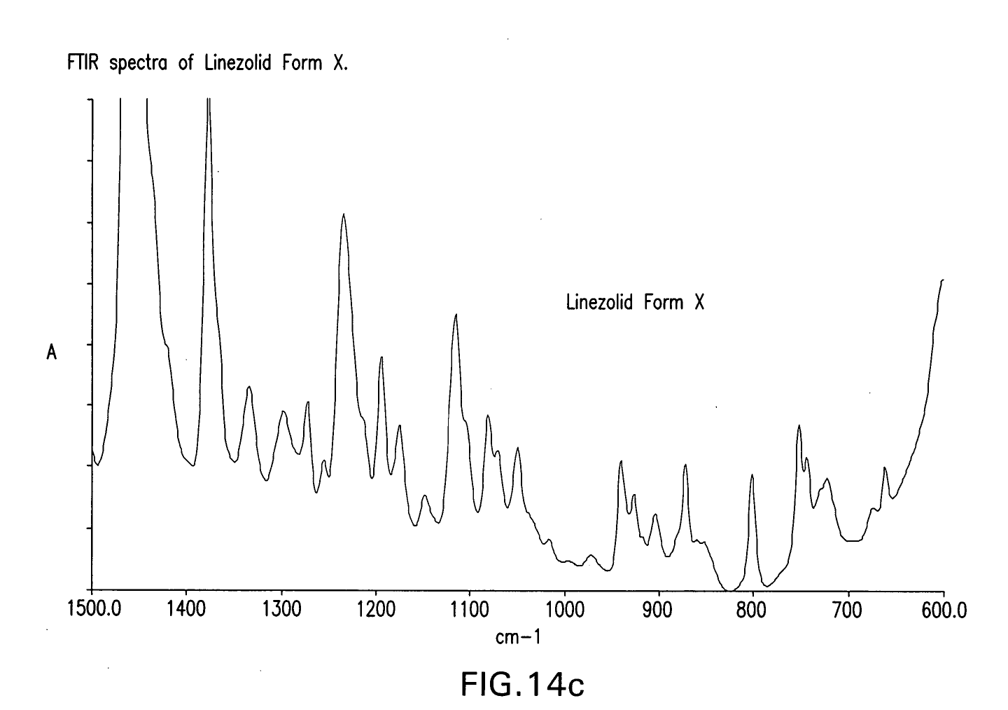

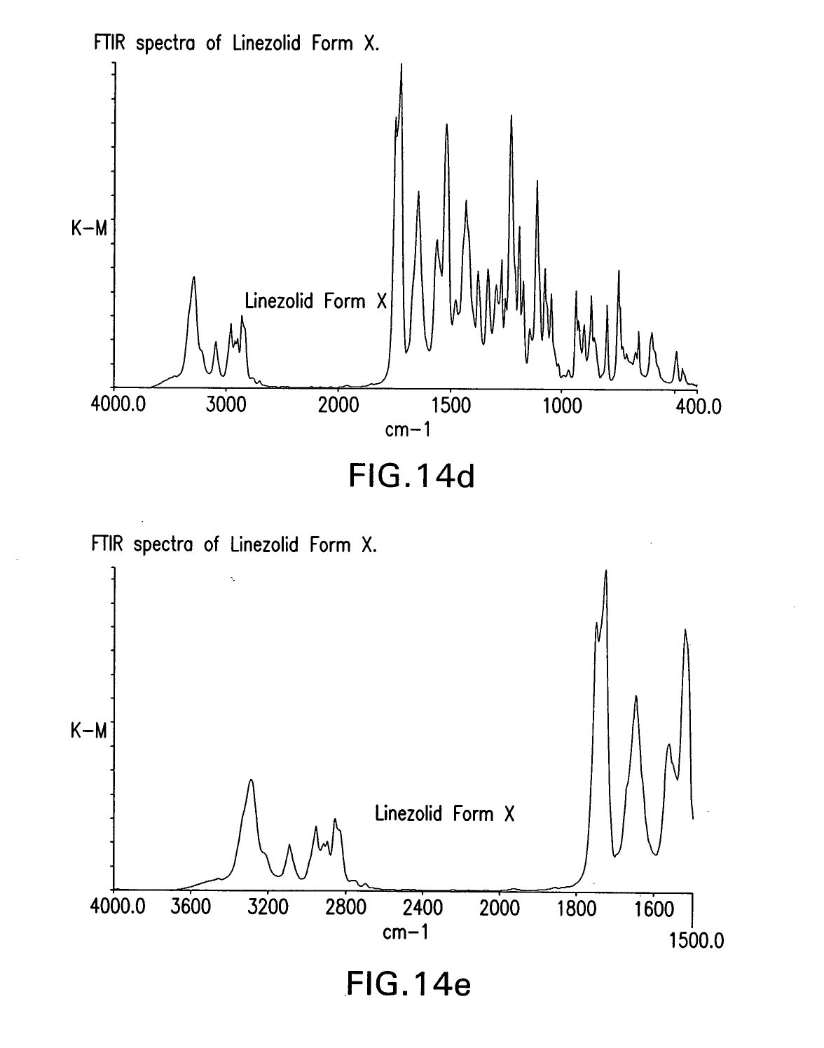

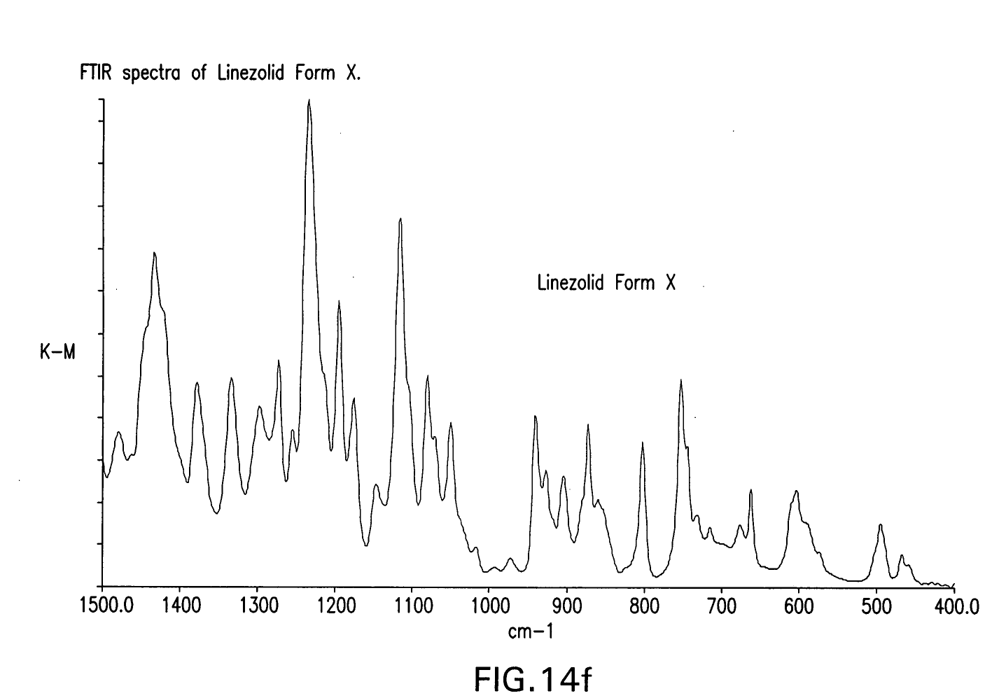

Crystalline Linezolid Form X may also be characterized by an FTIR spectrum with peaks at about 3090, 1524, 1335, 1195, 1115, 1081, 940, 927, 802, and 752 cm⁻¹. Crystalline Linezolid Form X may also be characterized by an FTIR spectrum substantially as depicted in FIG. 14a-14c or in FIG. 14d-14f.

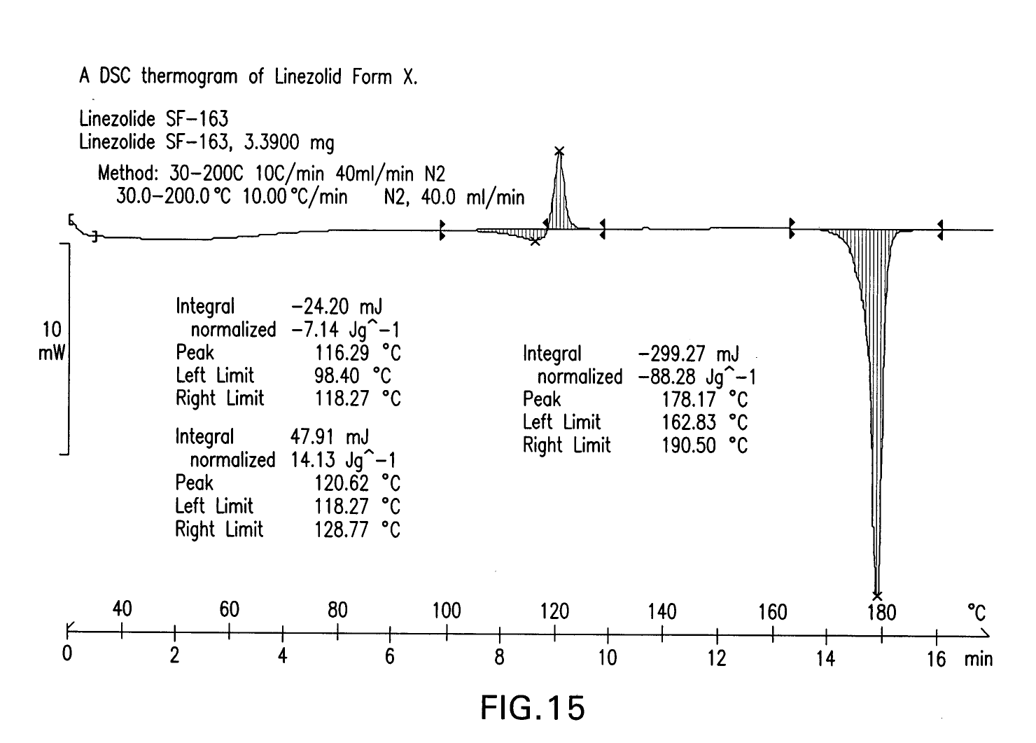

Crystalline Linezolid Form X may also be characterized by a DSC thermogram having a melting endotherm at around 115° C. and an exothermic peak at around 120° C. which probably represents its conversion into Form IV, substantially as depicted in FIG. 15.

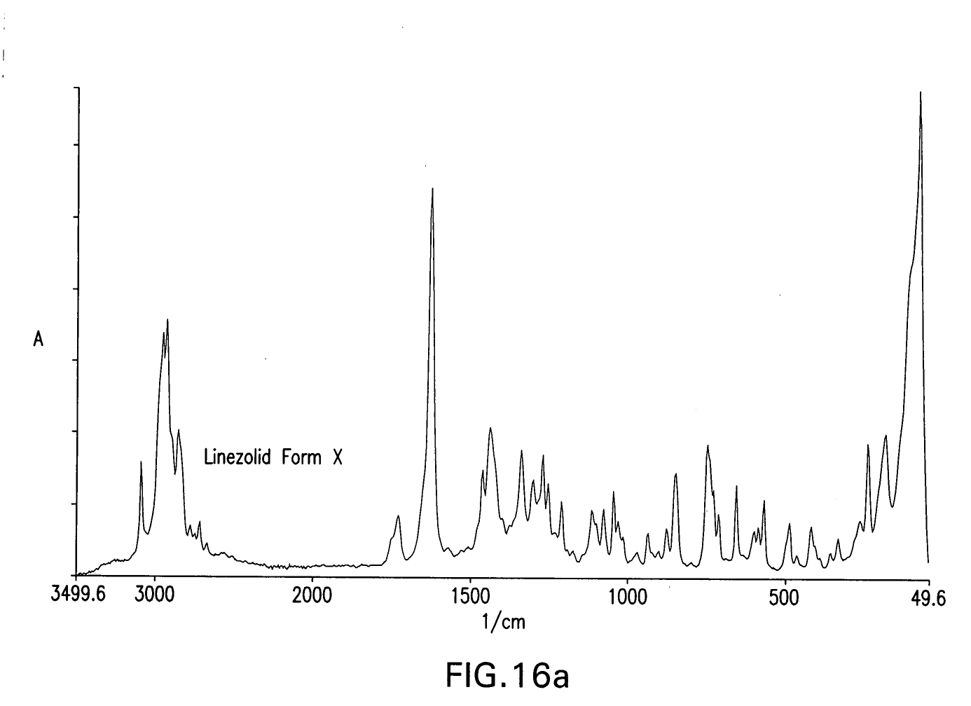

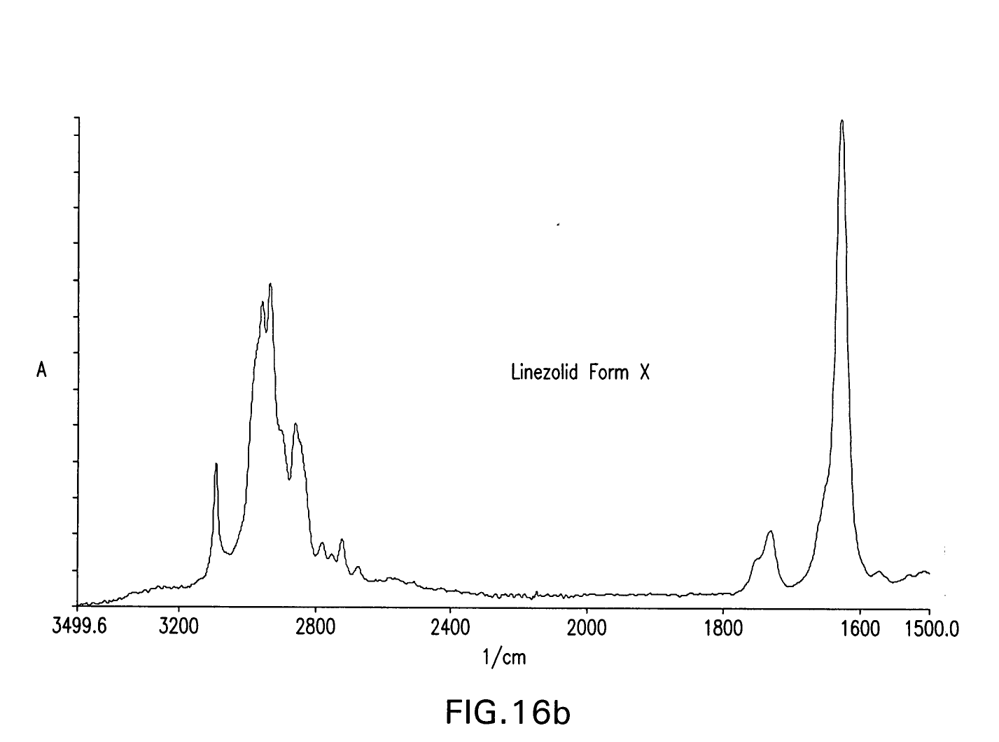

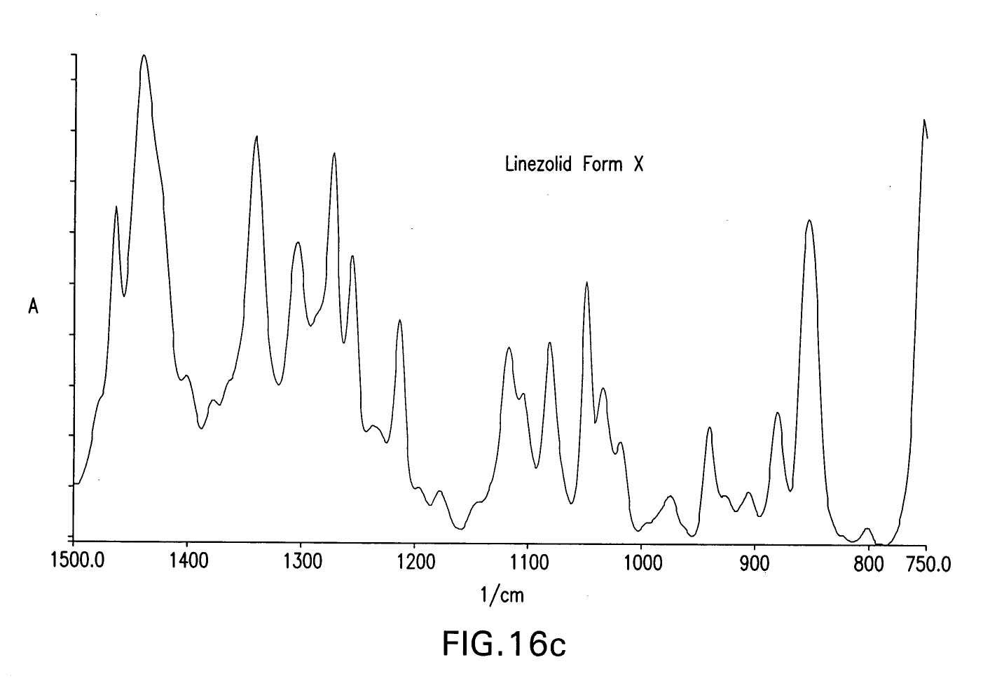

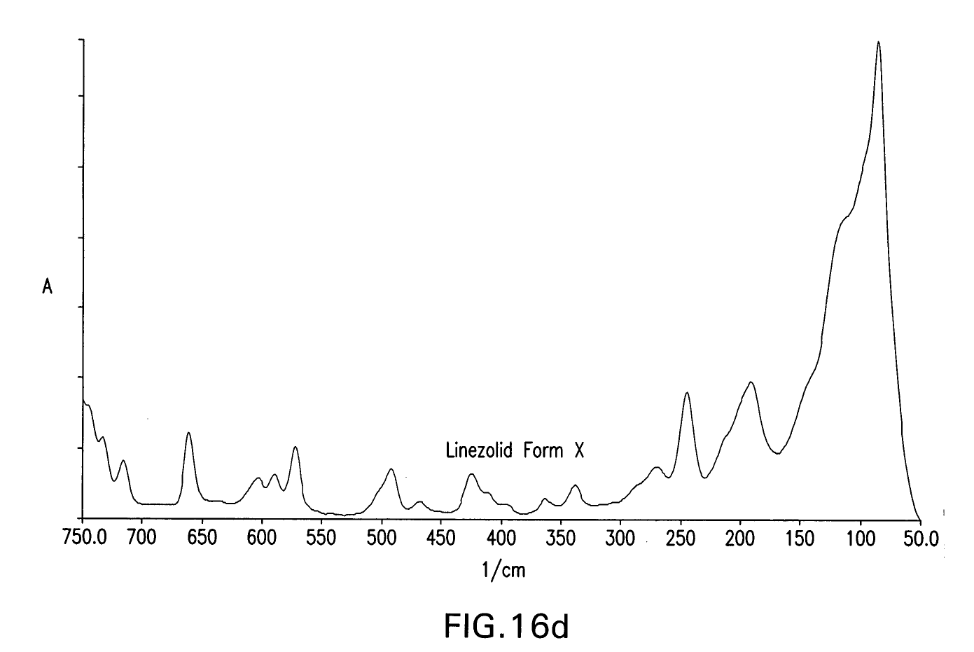

Crystalline Linezolid Form X may be further characterized by an FTRaman spectrum with peaks at about 2957, 2859, 880, 752 and 715 cm⁻¹. Crystalline Linezolid Form X may be further characterized by an additional peak in the FTRaman spectrum at about 975 cm⁻¹. Crystalline Linezolid Form X may be further characterized by an FTRaman spectrum substantially as depicted in FIG. 16a-16d.

Crystalline Linezolid Form X may be substantially free of Form II. Preferably, crystalline Linezolid Form X contains less than about 10%, preferably less than about 5%, and even more preferably less than about 1% (by weight) of Form II.

The invention provides crystalline Linezolid Form X, which has about 1-3% water by weight. Form X may also be characterized by a weight loss measured by thermal gravimetric analysis (TGA) of about 1-3% by weight. This embodiment of Form X is a hemihydrate. Thus, the present invention includes crystalline Linezolid Form X which has about 3% or less water by weight.

The stability of linezolid Form X was tested by storing it for 8 days at a relative humidity of between 0 and 100% at room temperature. The results are summarized in table 6.

TABLE 6

| Relative humidity [%] | Solvent content [%] | Crystal Form |
|---|---|---|
| As is | 1.9 (water content 1.9) | X |
| 0 | 1.6 | X |
| 20 | 1.5 | X |
| 40 | 1.4 | X |
| 60 | 2.0 | X |
| 80 | 2.4 | X |
| 100 | 0.7 | Form X + II |

Form X has proven to be stable between 0-80% RH. However at 100% RH, some of Form X converts to Form II.

Another aspect of the present invention is a process for preparing crystalline Linezolid Form X comprising:

a) dissolving Linezolid in water; and

b) lyophilizing the dissolved Linezolid to form crystalline Linezolid Form X.

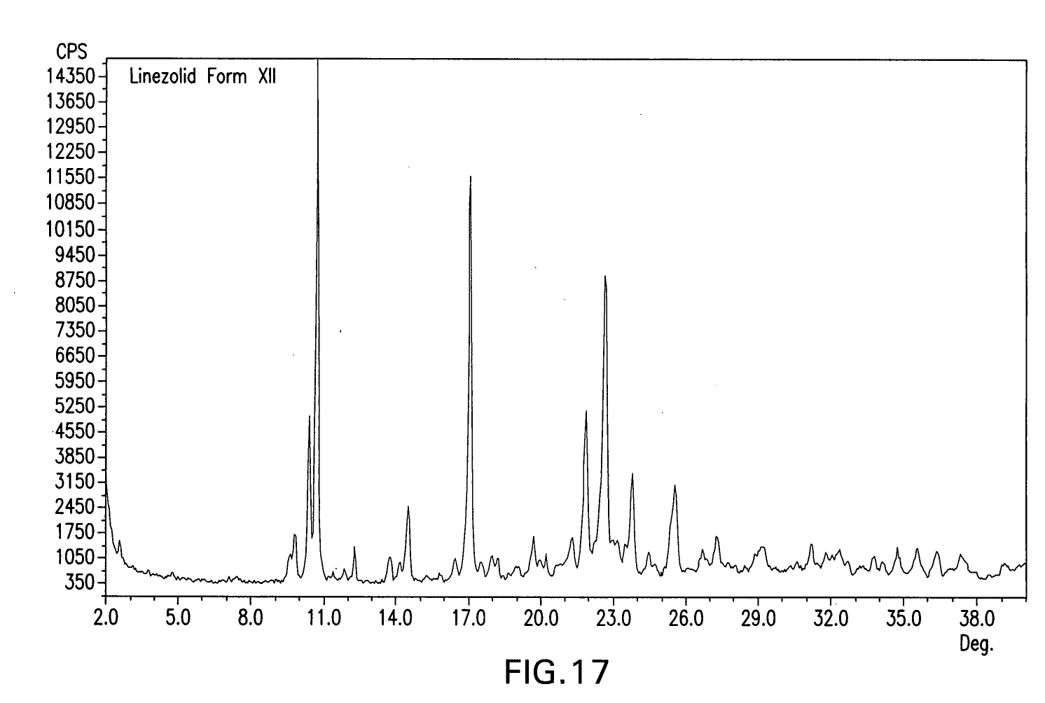

Another embodiment of the present invention is a crystalline Linezolid, (denominated Form XII), characterized by a PXRD pattern with peaks at about 10.4, 10.7,17.1, and 22.7±0.2 degrees 2 theta. Crystalline Linezolid Form XII may be further characterized by PXRD peaks at about 14.5, 21.9, and 23.8±0.2 degrees 2 theta. Crystalline Linezolid Form XII may be further characterized by a PXRD pattern substantially as depicted in FIG. 17.

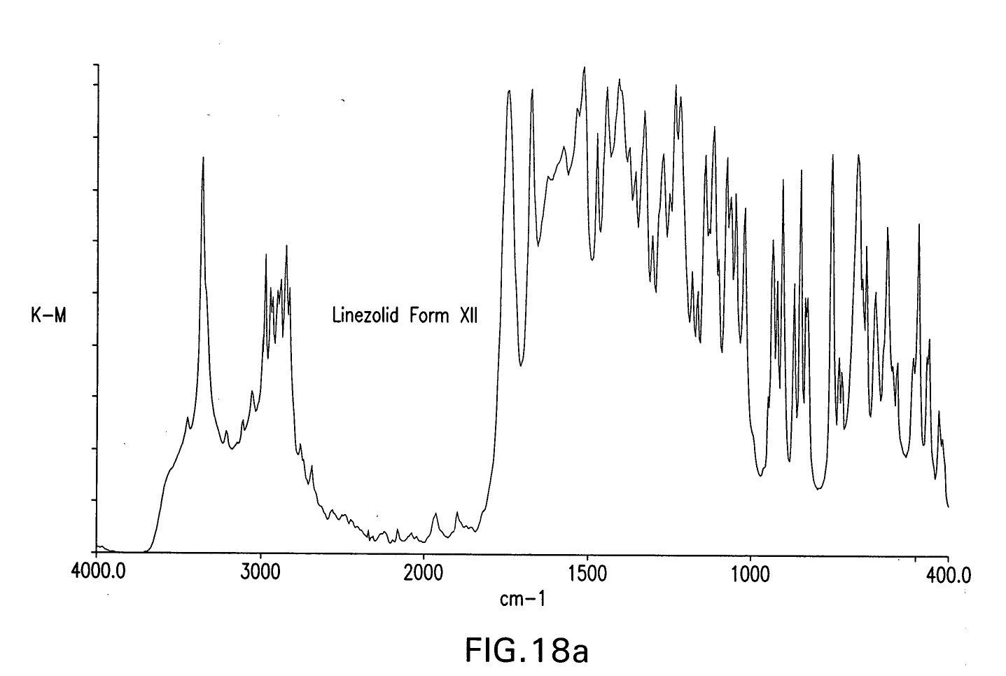

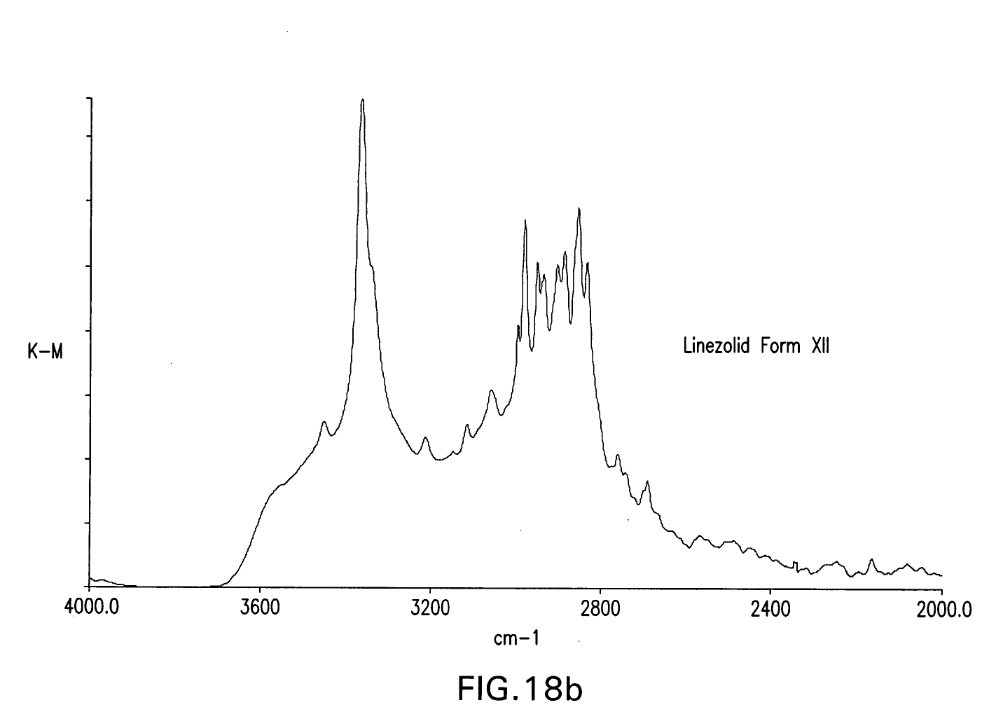

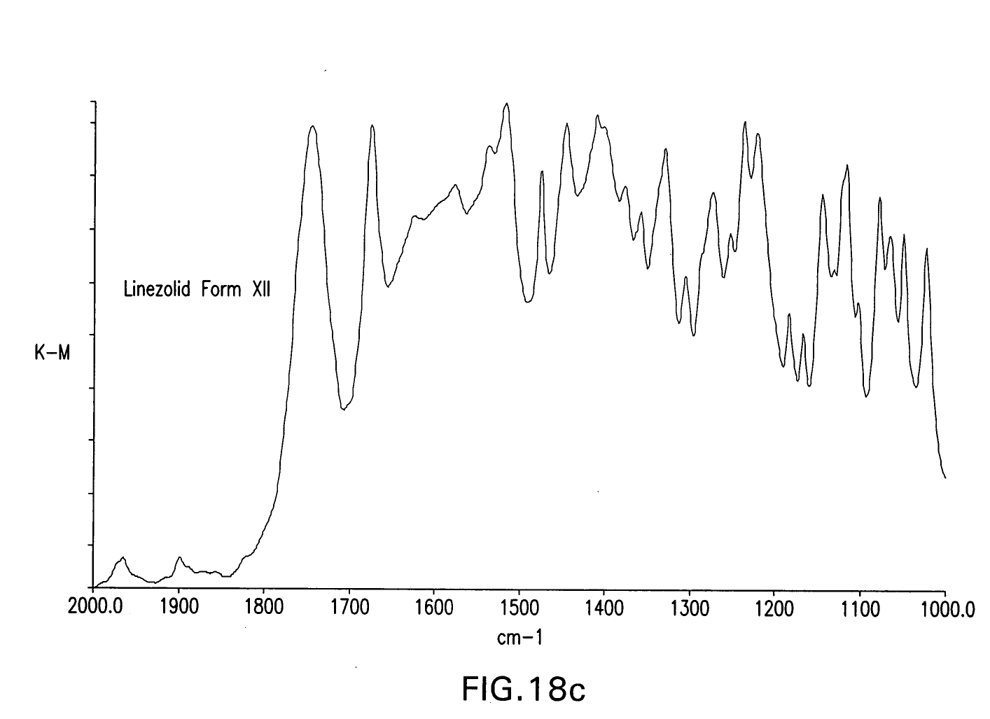

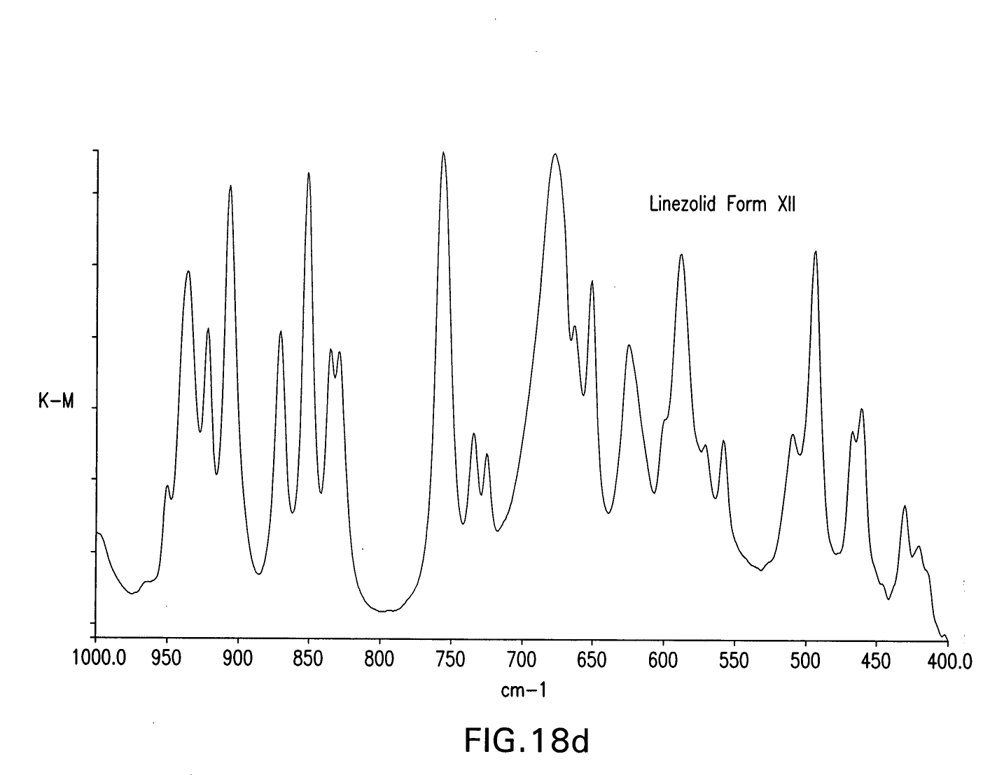

Crystalline Linezolid Form XII may be further characterized by an FTIR spectrum substantially as depicted in FIG. 18a-18d.

Crystalline Linezolid Form XII may be substantially free of Form II. Preferably, crystalline Linezolid Form XII contains less than about 10%, preferably less than about 5%, and even more preferably less than about 1% (by weight) of Form II.

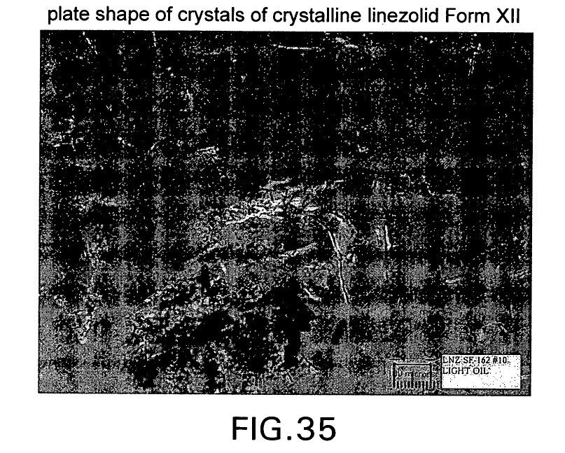

Crystalline linezolid Form XII is further characterized by its crystals having a plate shape, substantially as depicted in FIG. 35.

Another aspect of the present invention is a process for obtaining crystalline Linezolid Form XII comprising:
a) combining (S)—N-(4-morpholinyl-3-fluorophenyl)-2-oxo-5-oxazolidinyl-methyl amine, ethyl acetate, and a base to obtain a mixture;
b) cooling the mixture to a temperature of between −10° C. and +10° C.;
c) adding an acetylating agent to the solution;
d) maintaining the mixture produced in step (c) until Linezolid Form XII precipitates from the solution; and
e) recovering the precipitated Linezolid Form XII.

In the process for obtaining a precipitate of crystalline Linezolid of Form XII, the base may be selected from the group consisting of triethyl amine and pyrimidine.

In the process for obtaining a precipitate of crystalline Linezolid of Form XII, the acetylating agent may be selected from acetic anhydride and acetyl chloride.

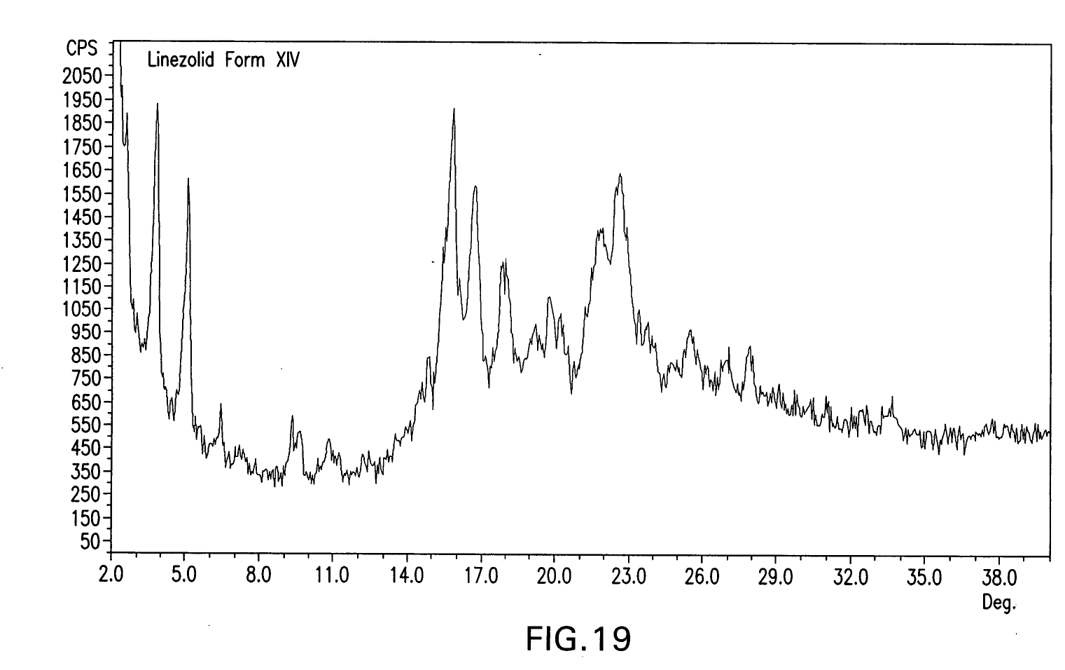

Another embodiment of the present invention is a crystalline Linezolid, (denominated Form XIV), characterized by a PXRD pattern with peaks at about 3.7, 5.0, 15.8, and 16.7±0.2 degrees 2 theta. Crystalline Linezolid Form XIV may be further characterized by a PXRD pattern substantially as depicted in FIG. 19.

Crystalline Linezolid Form XIV may be substantially free of Form II. Preferably, crystalline Linezolid Form XIV contains less than about 10%, preferably less than about 5%, and even more preferably less than about 1% (by weight) of Form II.

Another aspect of the present invention is a process for obtaining crystalline Linezolid Form XIV comprising:
a) combining (S)—N-(4-morpholinyl-3-fluorophenyl)-2-oxo-5-oxazolidinyl-methyl amine, ethyl acetate, and a base to obtain a reaction mixture;
b) adding an acetylating agent to the solution;
c) maintaining the reaction mixture at room temperature for at least 12 hours;
d) adding a water-immiscible solvent to obtain a precipitate of Linezolid Form XIV; and
e) recovering the precipitated Linezolid Form XIV.

In the process for obtaining a precipitate of crystalline Linezolid of Form XIV, the base may be selected from the group consisting of an organic base and an inorganic base. The organic base may be selected from triethyl amine and pyrimidine.

In the process for obtaining a precipitate of crystalline Linezolid of Form XIV, the acetylating agent may be selected from acetic anhydride and acetyl chloride.

In the process for obtaining a precipitate of crystalline Linezolid of Form XIV, the water-immiscible antisolvent may be an ether or an alkane. The ether may be selected from: diethyl ether, diisopropyl ether, methyl-t-butyl ether, petroleum ether and dipropyl ether. The alkane may be selected from hexane and heptane.

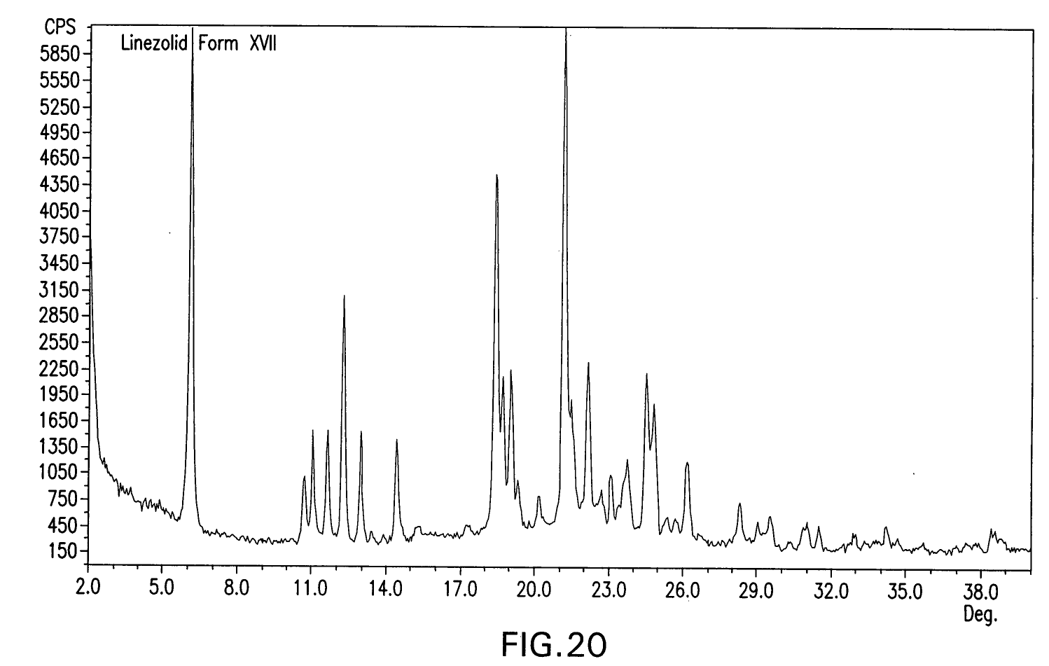

Another embodiment of the present invention is a crystalline Linezolid (denominated Form XVII), characterized by a PXRD pattern with peaks at about 6.1, 12.3, 18.4, and 21.2±0.2 degrees 2 theta. Crystalline Linezolid Form XVII may be further characterized by PXRD peaks at about 11.0, 11.7, 13.0, 14.4, and 22.2±0.2 degrees 2 theta. Crystalline Linezolid Form XVII may be further characterized by a PXRD pattern substantially as depicted in FIG. 20.

Crystalline Linezolid Form XVII may be substantially free of Form II. Preferably, crystalline Linezolid Form XVII contains less than about 10%, preferably less than about 5%, and even more preferably less than about 1% (by weight) of Form II.

Another aspect of the present invention is a process for obtaining crystalline Linezolid Form XVII comprising:
a) providing a solution of (S)—N-(4-morpholinyl-3-fluorophenyl)-2-oxo-5-oxazolidinyl-methyl amine in toluene at a temperature of about 3° C.;
b) adding an acetylating agent to the solution, and bringing the solution to a temperature of about 21° C. to obtain a precipitate of Linezolid Form XVII; and
c) recovering the precipitated Linezolid Form XVII.

In the process for obtaining a precipitate of crystalline Linezolid of Form XVII, the acetylating agent may be selected from acetic anhydride and acetyl chloride.

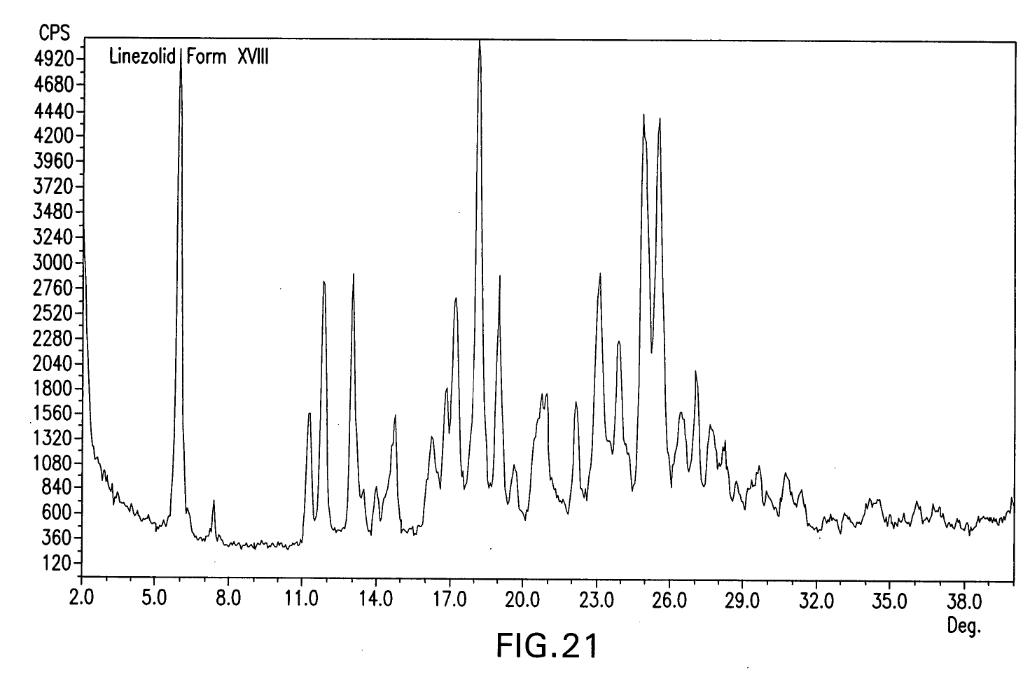

Another embodiment of the present invention is a crystalline Linezolid (denominated Form XVIII), characterized by a PXRD pattern with peaks at about 6.0, 11.8, 17.2, 18.2, and 24.9±0.2 degrees 2 theta. Crystalline Linezolid Form XVIII may be further characterized by PXRD peaks at about 11.3, 13.0, 19.0, 23.1, and 25.5±0.2 degrees 2 theta. Crystalline Linezolid Form XVIII may be further characterized by a PXRD pattern substantially as depicted in FIG. 21.

Crystalline Linezolid Form XVIII may be substantially free of Form II. Preferably, crystalline Linezolid Form XVIII contains less than about 10%, preferably less than about 5%, and even more preferably less than about 1% (by weight) of Form II.

Another aspect of the present invention is a process for obtaining crystalline Linezolid Form XVIII comprising:
a) providing, at a temperature of about 3° C. a solution of (S)—N-(4-morpholinyl-3-fluorophenyl)-2-oxo-5-oxazolidinyl-methyl amine in toluene and in the presence of a base to obtain a mixture;
b) adding an acetylating agent to the mixture;
c) bringing the mixture to a temperature of about 21° C. to obtain a precipitate of Linezolid Form XVIII.

In the process for obtaining a precipitate of crystalline Linezolid of Form XVIII, the base may be selected from the group consisting of an organic base and an inorganic base. The organic base may be selected from triethyl amine and pyrimidine.

In the process for obtaining a precipitate of crystalline Linezolid of Form XVIII, the acetylating agent may be selected from acetic anhydride and acetyl chloride.

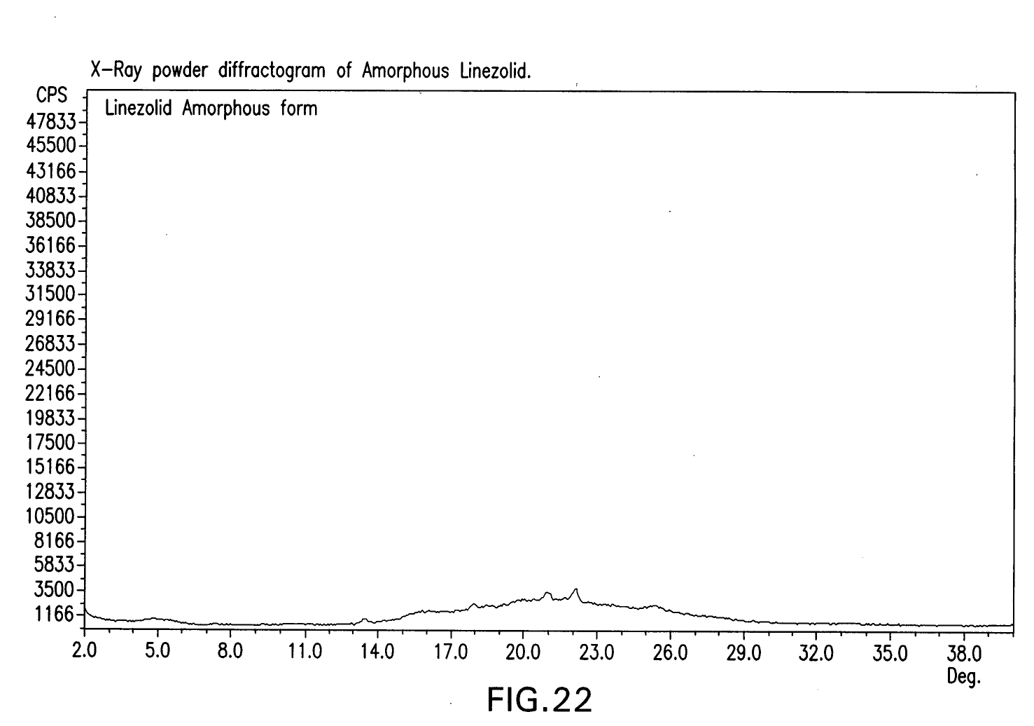

Yet another embodiment of the present invention is the amorphous form of Linezolid, characterized by a PXRD pattern that is substantially free of visible diffraction peaks, substantially as depicted in FIG. 22.

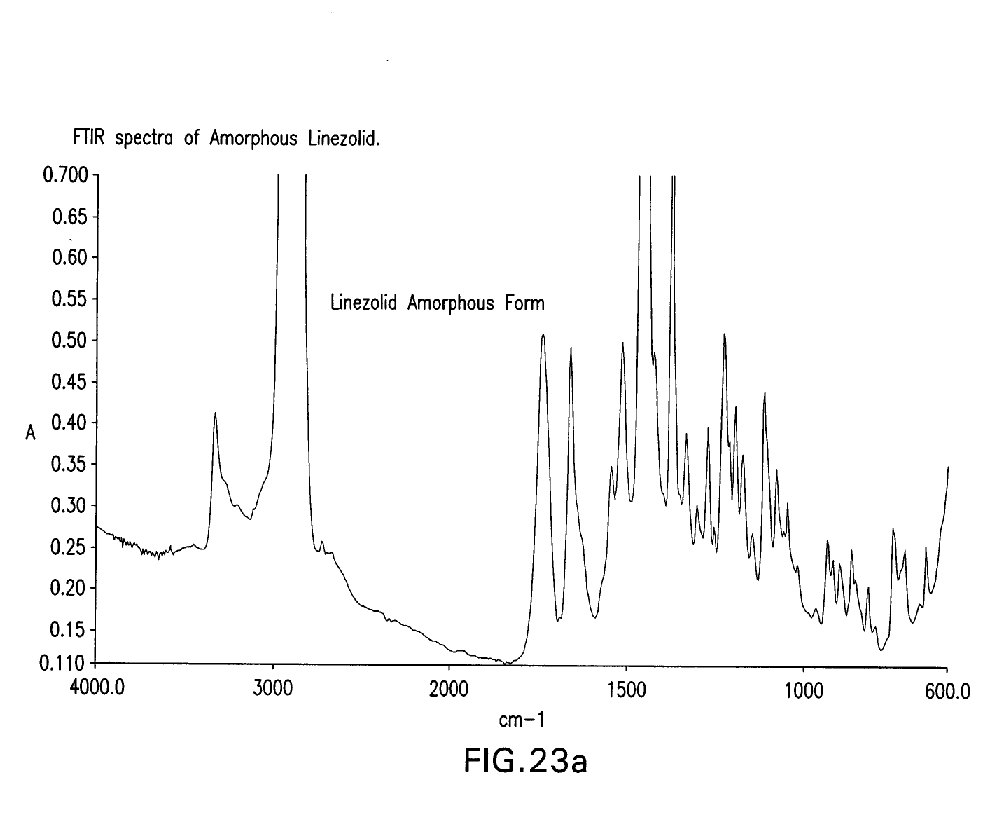

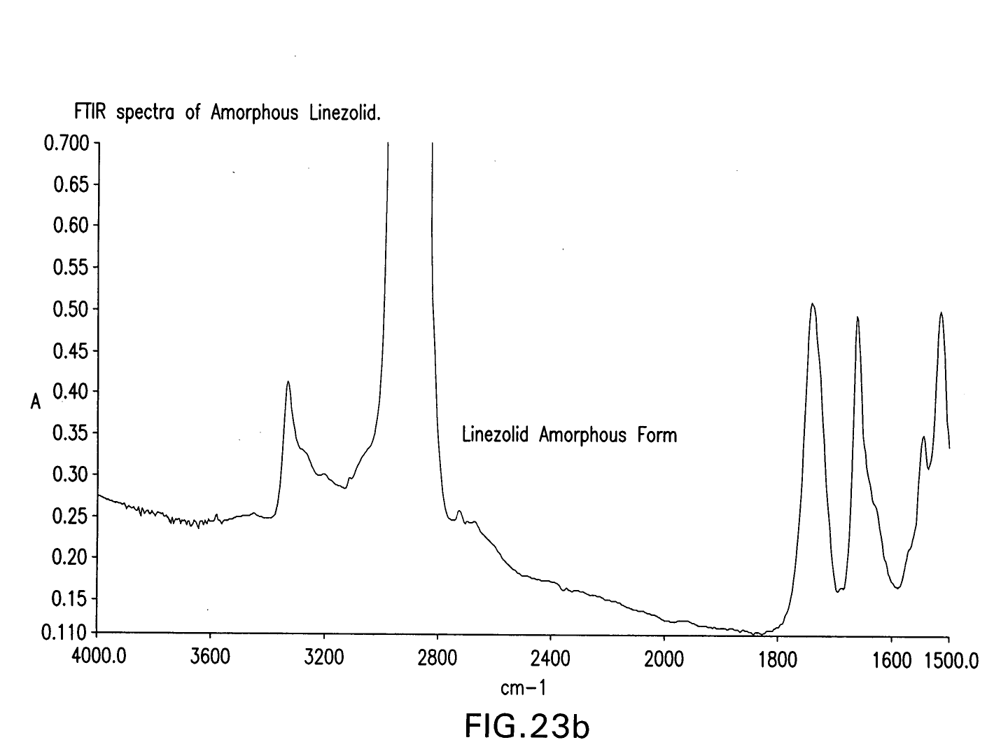

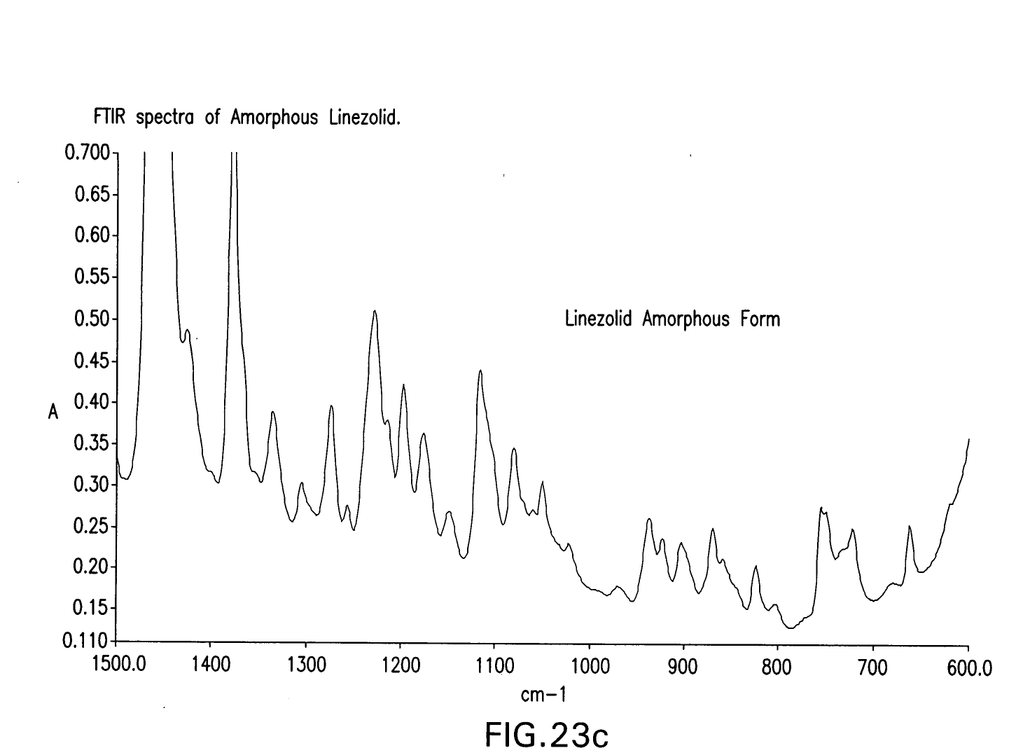

Amorphous Linezolid may also be characterized by FTIR peaks at about 1741, 1662, 1547, 1516, 1335, 1257, 1228, 1214, 1149, 1080, 1059, 1050, 903, 824, and 755 cm⁻¹. Amorphous Linezolid may also be characterized by an FTIR spectrum substantially as depicted in FIG. 23a-23c.

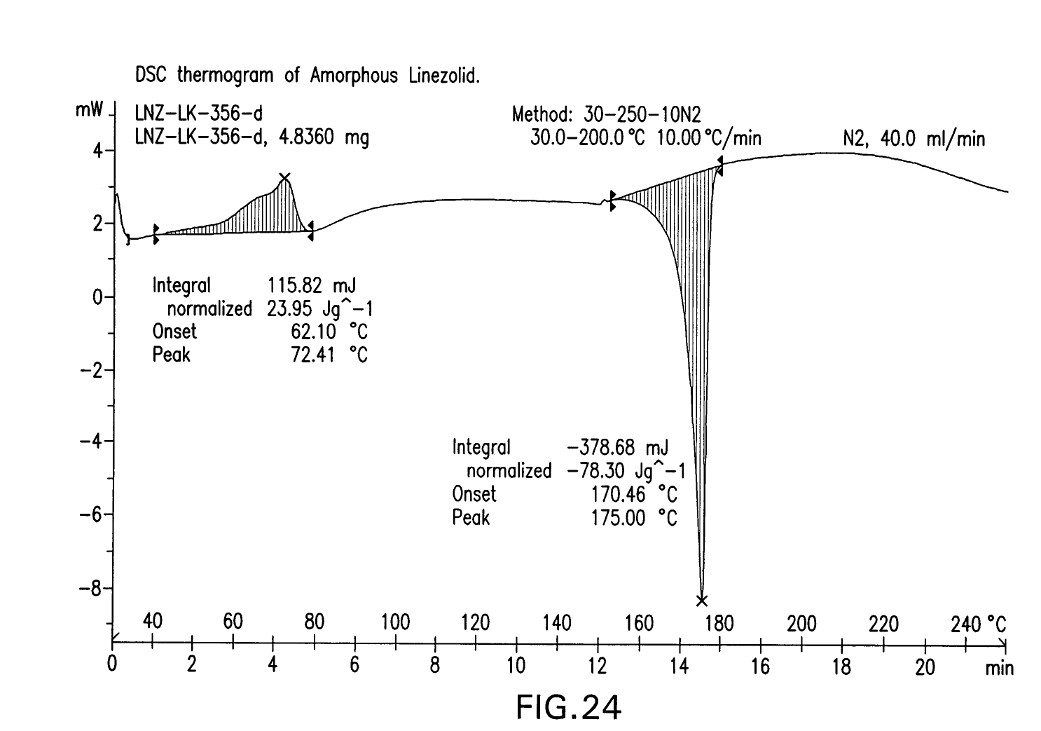

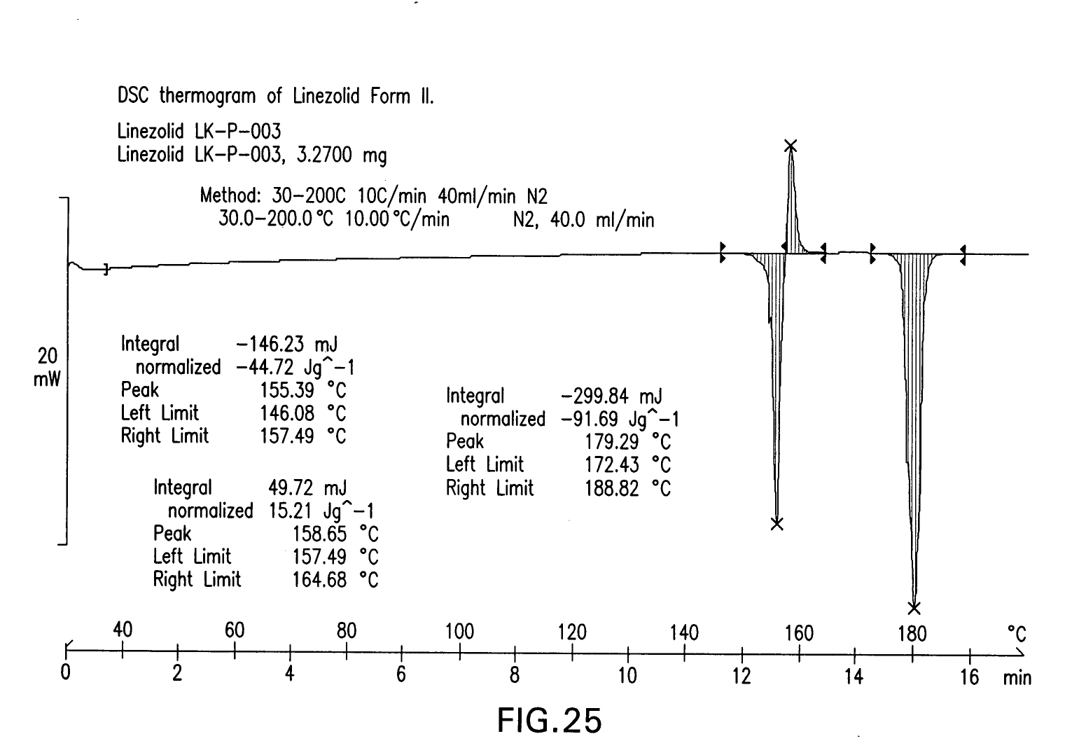

Amorphous Linezolid may also be characterized by a DSC thermogram having a broad exothermic peak around 70° C., followed by an endothermic peak at around 180° C., which corresponds to the final melting of Linezolid, substantially as depicted in FIG. 24.

The amorphous Linezolid of the present invention preferably contains less than 20% by weight Linezolid Form II, more preferably less than 10%, and even more preferably less than 5%.

The present invention also provides a process for preparing amorphous Linezolid, comprising the steps of:

a) melting crystalline Linezolid; and

b) cooling the melted Linezolid;

c) recovering amorphous Linezolid.

In the process for preparing amorphous Linezolid, the melting may be done by warming to about 180° C. The cooling of the melted Linezolid may be immediate, as, e.g., by transfer to a cool reservoir, or the cooling may be gradual to room temperature without external cooling. In the process for preparing amorphous Linezolid, the Linezolid used may be crystalline Linezolid Form II.

The amorphous form may also be obtained by heating linezolid Form X to a temperature of about 60° C. for 1.5 to 2 hours.

The crystalline and amorphous forms described above may be recovered by any process known in the art, such as filtration, concentration and evaporation.

The conditions may also be changed to induce precipitation. A preferred way of inducing precipitation is to reduce the solubility of the solvent. The solubility of the solvent may be reduced, for example, by cooling the solvent. Precipitation may also be induced by evaporating some of the solvent or by adding an anti-solvent.

The various crystalline forms of the present invention may be distinguished by their PXRD patterns. The crystalline forms have characteristic PXRD peak positions in the range of 2-40 degrees two theta. According to these characteristic peak positions, the skilled artisan can identify the crystalline forms and also identify and quantify their crystalline form impurities.

One skilled in the art would appreciate that there is a small amount of uncertainty involved in PXRD measurements, generally on the order of about ±0.2 degrees 2 theta for each peak. Accordingly, PXRD peak data herein are presented in the form of “a PXRD pattern with peaks at about A, B, C, etc. ±0.2 degrees 2 theta.” This indicates that, for the crystalline form in question, the peak at A could, in a given instrument on a given run, appear somewhere between A ±0.2 degrees 2 theta, the peak at B could appear at B ±0.2 degrees 2 theta, etc. Such small, unavoidable uncertainty in the identification of individual peaks does not translate into uncertainty with respect to identifying individual crystalline forms since it is generally the particular combination of peaks within the specified ranges, not any one particular peak, that serves to unambiguously identify crystalline forms.

The particle size distribution (PSD) of the active ingredient is one of the key parameters of a formulation. For measuring particle size, the following main methods may be employed: sieves, sedimentation, electrozone sensing (coulter counter), microscopy, Low Angle Laser Light Scattering (LALLS). The new forms of the invention have a maximum particle size of up to 500 μm. Preferably, the particle size is up to 300 μm. More preferably, the particle size is up to 200 μm. Even more preferably, the particle size is up to 100 μm. Most preferably, the particle size is up to 50 μm.

Another embodiment of the present invention is a pharmaceutical formulation comprising a therapeutically effective amount of a Linezolid crystalline form selected from the group consisting of Form TIII, Form V, Form VI, Form IX, Form X, Form XII, Form XIV, Form XVII, Form XVIII and amorphous, combined with a pharmaceutically acceptable excipient or carrier.

Another embodiment of the present invention is a method for treating a patient suffering from a gram positive bacterial infection, comprising the step of administering to the patient a pharmaceutical formulation comprising a therapeutically effective amount of a Linezolid selected from the group consisting of Form TIII, Form V, Form VI, Form IX, Form X, Form XII, Form XIV, Form XVII, Form XVIII and amorphous.

In a particular embodiment, the present invention provides a pharmaceutical formulation comprising crystalline Linezolid forms, having less than about 10%, preferably less than about 5%, and even more preferably less than about 1% (by weight) of Form II.

Alternatively, pharmaceutical formulations of the present invention may also contain one of the novel crystalline forms of Linezolid disclosed herein in a mixture with other forms of Linezolid.

In addition to the active ingredient(s), the pharmaceutical formulations of the present invention may contain one or more excipients. Excipients are added to the formulation for a variety of purposes.

Diluents may be added to the formulations of the present invention. Diluents increase the bulk of a solid pharmaceutical composition, and may make a pharmaceutical dosage form containing the composition easier for the patient and care giver to handle. Diluents for solid compositions include, for example, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON®, PLASDONE®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

The present invention is not intended to encompass true solutions of Linezolid whereupon the crystal structure of the novel crystalline forms and the properties that characterize the novel crystalline forms of Linezolid of the present invention are lost. However, the use of the novel forms to prepare such solutions (e.g., so as to deliver Linezolid in a liquid pharmaceutical formulation) is considered to be within the contemplation of the invention.

In liquid pharmaceutical compositions prepared using the crystalline forms of the present invention, Linezolid and any other solid excipients are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition may also contain a buffer such as guconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage of GEODON may be used as guidance. The oral dosage form of the present invention is preferably in the form of an oral capsule or tablet having a dosage of about 10 mg to about 160 mg, more preferably from about 20 mg to about 80 mg, and most preferably capsules or tablets of 20, 40, 60 and 80 mg. Daily dosages may include 1, 2, or more capsules per day.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The active ingredient and excipients may be formulated into compositions and dosage forms according to methods known in the art.

The crystalline forms of the present invention may be used in pharmaceutical formulations or compositions as single components or mixtures together with other crystalline forms of Linezolid or with amorphous Linezolid. However, it is preferred that the pharmaceutical formulations or compositions of the present invention contain 25-100% by weight, especially 50-100% by weight, of at least one of the novel forms, based on the total amount of Linezolid in the formulation or composition. Preferably, such an amount of the novel crystalline form of Linezolid is 75-100% by weight, especially 90-100% by weight. Highly preferred is an amount of 95-100% by weight.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

Instrumentation

X-Ray powder diffraction data were obtained by methods known in the art using a SCINTAG® powder X-ray diffractometer model X'TRA® equipped with a solid state detector. Copper radiation of 1.5418 Å was used. A round aluminum sample holder with round zero background quartz plate was used, with cavity of 25(diameter)*0.5(depth) mm. The obtained characteristic peaks were in the range of 2-40 degrees two theta.

Detection of Linezolid Form II in other crystal forms was done by detecting strong peaks of Form II in the diffractogram of the sample. The most characteristic XRD peaks of Form II appeared at 9.5, 14.2, 16.8, 21.6, 22.4, 23.5 and 25.3±0.2 degrees two theta.

DSC analysis was done using a Mettler 821 Star^{c}. The weight of the samples was about 5 mg; the samples were scanned at a rate of 10° C./min from 30° C. to 320° C. The oven was constantly purged with nitrogen gas at a flow rate of 40 ml/min. Standard 70 μl alumina crucibles covered by lids with 1 hole were used. IR analysis was done using a Perkin Elmer SPECTRUM ONE FT-IR spectrometer in DRIFTt mode, or using mineral oil mull technique. The samples in the 4000-400 cm⁻¹ interval were scanned 16 times with 4.0 cm⁻¹ resolution.

The FTRaman analysis was performed by Bruker RFS-100/S Raman spectrometer. The scanning parameters were:
Range: 3500 to 50 cm⁻¹;
Aperture Setting: 10.0 mm;
Low Pass Filter 16: 1 kHz;
Source Setting Laser: 9394.0 cm⁻¹, 1600 mW;
Raman Laser Power: 500 mW;
Scanner: Velocity 5.0 at 4 kHz;
Sample Scans: 100; and
Resolution: 4.0 cm⁻¹.

### Example 1

### Preparation of Linezolid Form TIII by Crystallization of Linezolid

Linezolid Form II (2.0 g) was dissolved in dichloromethane (40 ml) at room temperature. The solution was washed with water (300 ml) and the phases were separated. The organic phase was evaporated to dryness to obtain crystals. The crystals were analyzed by PXRD and showed a novel form of Linezolid, Linezolid Form TIII.

### Example 2

### Preparation of Linezolid Form V

3 g of crude R—N-(4-morpholinyl-3-fluorophenyl)-2-oxo-5-oxazolidinyl-methyl amine was mixed with 100 ml ethyl acetate and 3 ml triethyl amine. The reaction mixture was cooled to 0° C. and 3 ml acetic anhydride was added dropwise. The reaction mixture was stirred for 1 hr and then the ice bath was removed. Petroleum ether was added and the mixture was stirred at RT during half an hour until precipitation occurred. The crystals were filtered and dried in a vacuum oven at −60° C. until constant weight. 3.17 g Linezolid was obtained. The crystals were analyzed by PXRD and FTIR, and showed a novel form of Linezolid (Form V).

### Example 3

### Preparation of Linezolid Form VI

3 g of R—N-(4-morpholinyl-3-fluorophenyl)-2-oxo-5-oxazolidinyl-methyl amine was prepared by standard hydrogenation using palladium over charcoal from the corresponding R—N-(4-morpholinyl-3-fluorophenyl)-2-oxo-5-oxazolidinyl-methyl azide. The amine was isolated by evaporation. Without further purification, 2 g of amine was mixed with 20 ml ethyl acetate and 2 ml triethyl amine. The reaction mixture was cooled to 0° C. and acetyl chloride was added (2 equivalents) dropwise. Linezolid was precipitated from the reaction mixture, filtered, and dried (vacuum oven −50° C.) until constant weight. The crystals were analyzed by PXRD and FTIR, and showed a novel form of Linezolid (Form VI).

### Example 4

### Preparation of Linezolid Form VII

10 g R—N-(3-fluoro-4-morpholinyl-phenyl)-2-oxo-5-oxazolidinyl-methylazide, 2.5 g NaBH₄, 1 g NaOH, 3.0 g Aliquot 336 (a commercially available phase transfer catalyst) were slurried in 50 ml ethyl-acetate and heated to reflux for 4 hr. The reaction mixture was cooled to RT. 5 ml Et₃N and 6 ml Ac₂O were added. The reaction was stirred at RT overnight, then filtered and washed with H₂O and EtOAc. The product was dried at 50° C. under vacuum to obtain 3.54 g of Form VII.

### Example 5

### Preparation of Linezolid Form IX

### Example 5a

### Preparation of (±)-1-Amino-3-chloro-2-propanol HCl

59.50 g benzaldehyde in 150 ml ethanol was cooled to 18° C. 120.77 ml ammonia (25%) was added, followed by 6 ml ethanol. 50 g (±) epichlorohydrin (commercially available from Aldrich) and 22 ml EtOH were added. The reaction mixture was stirred at 40° C. for 7 hr and then cooled to 20-21° C. for 13.5 hr. The solution was concentrated in vacuum to a volume of 133 ml. 115 ml toluene was added, and the solution was heated to 35° C. 94.5 ml HCl 32% and 77 ml water were added over 5 min. at 35° C.

The two-phases of the mixture were stirred at 45° C. for 3 hr. The phases were separated. The upper phase was washed with 28 ml water. The aqueous phases were combined and 28 ml ethanol was added. The mixture was concentrated to 95 ml. Ethanol 38×7 ml was added, and the mixture was concentrated to 95 ml after each addition. 95 ml EtOH was added. The slurry was warmed to reflux for ½ hr, cooled to RT, and then cooled to −25° C. for 18 hr under stirring. The solution was evaporated to dryness, and 59.2 g were obtained.

### Example 5b

### Preparation of (±)-N-[2-(Acetyloxy)-3-chloropropyl]acetamide

59 g of (±)-1-amino-3-chloro-2-propanol HCl (obtained from example 5a, 150 ml EtOAc and 75 ml triethylamine were stirred at RT. Then acetic anhydride (88 ml) was added. The reaction was stirred at RT overnight. The reaction mixture was cooled to 6° C. 70 ml water was added. The mixture was cooled to 0° C. 240 ml potassium carbonate (47%) was added dropwise. 150 ml H₂O and 70 ml ethyl acetate were added. The phases were separated. The organic phase was washed with a solution of sodium chloride (8 g/200 ml H₂O). The combined aqueous phase was stirred with methylene chloride (800 ml) at RT. After 48 hr, the organic phase was evaporated to dryness to give 18.9 g of (±)-N-[2-(acetyloxy)-3-chloropropyl]acetamide.

### Example 5c

### Preparation of (±)Linezolid Form IX

16 g of carbobenzoxy-3-fluoro-4-morpholinyl-aniline, 16.3 g of t-Boc and 48 ml THF were stirred at RT. 3.12 g methanol was added. 18.9 g of (±)-N-[2-(acetyloxy)-3-chloropropyl]acetamide (obtained in example 10b) in 20 ml THF was added. The solution was stirred at RT for 15 hr. 60 ml water, 60 ml methylene chloride, and 6 ml acetic acid were added to the solution and stirred at RT for 30 min. The phases were separated. The aqueous phase was washed with 100 ml methylene chloride. The combined organic phase was concentrated in vacuum to dryness. The resulting oil was slurried with 20 ml hexane at RT. The obtained crystals were filtered and dried under nitrogen to obtain 0.52 g of (±) Linezolid Form IX.

### Example 6

### Preparation of Linezolid Form X by lyophilization of Linezolid

2.0 g of Linezolid Form II was dissolved in 800 ml water, frozen at −50° C., and placed in a laboratory lyophilizer. The vacuum was set to 0.2 mm Hg. The water was evaporated during 5 days at a temperature of 10° C. The resulting material was analyzed by PXRD and showed a novel form of Linezolid (Form X).

### Example 7

### Preparation of Linezolid Form XII

A flask charged with a mixture containing 2.8 g of (S)—N-(4-morpholinyl-3-fluorophenyl)-2-oxo-5-oxazolidinyl-methyl amine in 40 ml ethyl acetate was stirred at 0° C. Triethyl amine (2 equivalents) was added followed by acetic anhydride (2.5 equivalents). The reaction mixture was maintained at 0° C. overnight. Linezolid precipitated and was filtered and dried in an oven at 50° C. The crystals were analyzed by PXRD and showed a novel form of Linezolid (Form XII).

### Example 8

### Preparation of Linezolid Form XIV

To a solution of 5.6 g crude (S)—N-(4-morpholinyl-3-fluorophenyl)-2-oxo-5-oxazolidinyl-methyl amine in 300 ml ethyl acetate was added 10 ml triethyl amine. The reaction mixture was stirred at 25° C. Acetic anhydride (10 ml) was added dropwise. The reaction mixture was stirred overnight at room temperature. Petroleum ether was added and a gelatinous precipitation was observed. The reaction mixture was stirred overnight at room temperature. Linezolid precipitated and the crystals were filtered. The wet crystals were analyzed by PXRD and showed a novel form of Linezolid (Form XIV).

### Example 9

### Preparation of Linezolid Form XVII

A flask charged with a solution containing 1.5 g of (S)—N-(4-morpholinyl-3-fluorophenyl)-2-oxo-5-oxazolidinyl-methyl amine in 60 ml toluene was cooled to 3° C. and acetic anhydride (2 equivalents) was added dropwise. The reaction mixture was brought to RT. Linezolid was precipitated from the reaction mixture and filtered. The wet crystals were analyzed by PXRD and showed a novel form of Linezolid (Form XVII).

### Example 10

### Preparation of Linezolid Form XVIII

To a flask charged with a mixture containing 1.5 g of (S)—N-(4-morpholinyl-3-fluorophenyl)-2-oxo-5-oxazolidinyl-methyl amine in 60 ml toluene at 25° C., triethyl amine (5.2 ml) was added. The mixture was cooled to 3° C. and acetic anhydride (2.5 equivalents) was added dropwise. The reaction mixture was brought to RT. Linezolid that precipitated from the reaction mixture was filtered. The wet crystals were analyzed by PXRD showing a novel form of Linezolid (Form XVII).

### Example 11

### Preparation of amorphous Linezolid by Melting of Linezolid

Linezolid Form II (2.0 g) was heated in a test tube until melting occurred. The liquefied material was transferred to a cooled reservoir. The solid form obtained was analyzed by PXRD and showed a novel form of Linezolid, the amorphous form.

### Example 12

### Preparation of amorphous Linezolid by heating of Linezolid

Linezolid Form X (0.5 g) was heated in a conventional oven at a temperature of 60° C. for 1.5-2 hours. The amorphous form obtained was analyzed by PXRD.

1. Linezolid hydrate.

2. Crystalline Linezolid and solvates thereof characterized by data selected from the group consisting of: an X-ray powder diffraction pattern having peaks at about 12.3, 17.6, 22.2, 24.6, and 31.8±0.2 degrees 2 theta, an FTIR spectrum having peaks at about 3336, 2497, 1742, 1662, 1546, 1516, 1425, 1229, and 1038 cm⁻¹ and an FTRaman spectrum having peaks at about 2933, 2978, 1082 and 1036 cm⁻¹.

3. The crystalline linezolid of claim 2, characterized by an X-ray powder diffraction pattern having peaks at about 12.3, 17.6, 22.2, 24.6, and 31.8±0.2 degrees 2 theta.

4. The crystalline linezolid of claim 3, further characterized by an X-ray powder diffraction pattern having peaks at about 7.5, 13.5, 21.1, 25.5, and 27.8±0.2 degrees 2 theta.

5. The crystalline linezolid of claim 4, characterized by an X-ray powder diffraction pattern substantially as depicted in FIG. 5.

6. The crystalline linezolid of claim 2, characterized an FTIR spectrum having peaks at about 3336, 2497, 1742, 1662, 1546, 1516, 1425, 1229, and 1038 cm⁻¹.

7. The crystalline linezolid of claim 6, characterized by an FTIR spectrum substantially as depicted in FIGS. 6a-6c.

8. The crystalline linezolid of claim 2, characterized an FTRaman spectrum having peaks at about 2933, 2978, 1082 and 1036 cm⁻¹.

9. The crystalline linezolid of claim 8, further characterized by an FTRaman spectrum having peaks at about 1660, 1428, 1465, 904, 661, 462, 424, 339 and 127 cm⁻¹.

10. The crystalline linezolid of claim 9, characterized by an FTRaman spectrum substantially as depicted in FIGS. 7a-7d.

11. The crystalline linezolid of claim 2, having plate-shaped crystals.

12. The crystalline linezolid of claim 2, containing less than about 10% of linezolid Form II.

13. A process for the preparation of the crystalline linezolid of claim 2 comprising:
a) dissolving R—N-(4-morpholinyl-3-fluorophenyl)-2-oxo-5-oxazolidinyl-methyl amine in a solution of ethyl acetate and a base;
b) cooling the solution;
c) adding an acetylating agent to the solution and maintaining the solution for at least one hour;
d) adding an anti-solvent so as to precipitate the Linezolid; and
e) recovering the precipitated Linezolid of claim 2.

14. The process of claim 13, wherein the base in step a) is triethyl amine.

15. The process of claim 13, wherein the acetylating agent in step c) is acetyl chloride or acetic anhydride.

16. The process of claim 13, wherein the anti-solvent in step d) is petroleum ether.

17. Crystalline Linezolid and solvates thereof characterized by data selected from the group consisting of: an X-ray powder diffraction pattern having peaks at about 4.7, 15.7, and 21.7±0.2 degrees 2 theta, an FTIR spectrum having peaks at about 3090, 1524, 1335, 1195, 1115, 1081, 940, 927, 802, and 752 cm⁻¹ and an FTRaman spectrum having peaks at about 2957, 2859, 880, 752 and 715 cm⁻¹.

18. The crystalline linezolid of claim 17, characterized by an X-ray powder diffraction pattern having peaks at about 4.7, 15.7, and 21.7±0.2 degrees 2 theta.

19. The crystalline linezolid of claim 18, further characterized by an X-ray powder diffraction pattern having peaks at about 3.5, 10.3, and 20.2 degrees 2 theta.

20. The crystalline linezolid of claim 19, characterized by an X-ray powder diffraction pattern substantially as depicted in FIG. 13.

21. The crystalline linezolid of claim 17, characterized an FTIR spectrum having peaks at about 3090, 1524, 1335, 1195, 1115, 1081, 940, 927, 802, and 752 cm⁻¹.

22. The crystalline linezolid of claim 21, characterized by an FTIR spectrum substantially as depicted in FIGS. 14a-14c.

23. The crystalline linezolid of claim 17, characterized an FTRaman spectrum having peaks at about 2957, 2859, 880, 752 and 715 cm⁻¹.

24. The crystalline linezolid of claim 23, further characterized by an FTRaman spectrum having a peak at about 975 cm⁻¹.

25. The crystalline linezolid of claim 24, characterized by an FTRaman spectrum substantially as depicted in FIGS. 16a-16d.

26. The crystalline linezolid of claim 17, containing less than about 10% of linezolid Form II.

27. A process for the preparation of the crystalline linezolid of claim claim 17 comprising:
a) dissolving linezolid in water; and
b) lyophilizing the dissolved linezolid to form the crystalline Linezolid of claim 17.

28. Crystalline Linezolid forms and hydrates thereof characterized by having peaks at X-ray powder diffraction patterns selected from the group consisting of:
13.5, 16.8, 21.1, 21.7, and 22.2±0.2 degrees 2 theta;
12.3, 21.3, 24.7, 25.2, and 27.7±0.2 degrees 2 theta;
13.4, 17.9, 21.4, 22.3, and 25.6±0.2 degrees 2 theta;
10.4, 10.7, 17.1, and 22.7±0.2 degrees 2 theta;
3.7, 5.0, 15.8, and 16.7±0.2 degrees 2 theta;
6.1, 12.3, 18.4, and 21.2±0.2 degrees 2 theta; or
6.0, 11.8, 17.2, 18.2, and 24.9±0.2 degrees 2 theta.

29. The crystalline linezolid forms and hydrates of claim 28, containing less than about 10% of linezolid Form II.

30. A pharmaceutical composition prepared by combining at least one pharmaceutically-acceptable excipient with at least one of the crystalline forms, and hydrates of Linezolid of any one of claims 2-12, 17-26, 28, and 29.

31. Amorphous Linezolid characterized by data selected from the group consisting of:
a) an X-ray powder diffraction pattern that is substantially free of visible diffraction peaks;
b) an X-ray powder diffraction pattern substantially as shown in FIG. 22;
c) an FTIR spectrum having peaks at about 1741, 1662, 1547, 1516, 1335, 1257, 1228, 1214, 1149, 1080, 1059, 1050, 903, 824, and 755 cm⁻¹;
d) an FTIR spectrum substantially as shown in FIG. 23a-23c;
e) a DSC thermogram having a broad exothermic peak around 70° C., followed by an endothermic peak at around 180° C.; and
f) a DSC thermogram substantially as shown in FIG. 24.

32. The amorphous Linezolid of claim 1 containing less than 20% by weight Linezolid Form II.

33. The amorphous Linezolid of claim 1 containing less than 10% by weight Linezolid Form II.

34. The amorphous Linezolid of claim 1 containing less than 5% by weight Linezolid Form II.

35. A pharmaceutical formulation comprising a therapeutically effective amount of the amorphous Linezolid of claim 1 and a pharmaceutically acceptable excipient.
